(19) United States
(12) Patent Application Publication
(10) Pub. No.: US 2025/0213496 A1
(43) Pub. Date: Jul. 3, 2025

(54) LIPID NANOPARTICLES AND PREPARATION METHODS AND USE THEREOF

(71) Applicants: INSTITUTE OF CHEMISTRY, CHINESE ACADEMY OF SCIENCES, Beijing (CN); PROXYBIO THERAPEUTICS CO., LTD., Shenzhen (CN)

(72) Inventors: Xueguang LV, Beijing (CN); Jiaqi LIN, Shenzhen (CN); Wei LI, Beijing (CN); Jingjiao LI, Beijing (CN); Xingdi CHENG, Beijing (CN)

(73) Assignees: INSTITUTE OF CHEMISTRY, CHINESE ACADEMY OF SCIENCES, Beijing (CN); PROXYBIO THERAPEUTICS CO., LTD., Shenzhen (CN)

(21) Appl. No.: 19/079,425

(22) Filed: Mar. 13, 2025

Related U.S. Application Data
(63) Continuation-in-part of application No. PCT/CN2023/116854, filed on Sep. 4, 2023.

(30) Foreign Application Priority Data
Oct. 11, 2022 (CN) 202211243350.1

(51) Int. Cl. A61K 9/51; A61K 39/00; A61K 39/12; A61K 39/135; A61K 39/145; A61K 39/21; A61K 39/215; A61K 47/12; A61K 47/24

(52) U.S. Cl. CPC A61K 9/5123; A61K 9/5146; A61K 9/5192; A61K 39/0011; A61K 39/12; A61K 39/135; A61K 39/145; A61K 39/21; A61K 39/215; A61K 47/12; A61K 47/24

(57) ABSTRACT

A lipid nanoparticle and a preparation method and use thereof are provided. The lipid nanoparticle (LNP) includes a carrier and an encapsulated nucleic acid, the carrier includes an ionizable lipid, a helper phospholipid, a PEGylated lipid, cholesterol and its derivatives, and a retinoid compound; and the nucleic acid is one or more of mRNA, circRNA, siRNA, microRNA, antisense nucleic acid, and plasmid.

### CROSS-REFERENCE TO RELATED APPLICATIONS

The present application is a continuation-in-part application of International Application No. PCT/CN2023/116854, filed on Sep. 4, 2023, which claims priority to Chinese Patent Application No. 202211243350.1, filed on Oct. 11, 2022, the entire contents of each of which are incorporated herein by reference.

### TECHNICAL FIELD

The present disclosure relates to the field of pharmaceutical technology, and in particular, to a five-component lipid nanoparticle (LNP) that can encapsulate both nucleic acid drugs/vaccines and retinoid compounds and a preparation method and use thereof.

### BACKGROUND

After the outbreak of the COVID-19 pandemic, the U.S. Food and Drug Administration approved messenger RNA (mRNA) COVID-19 vaccines from two companies, Moderna and Pfizer-BioNTech, in record time, giving mRNA vaccines unprecedented attention. Compared with traditional protein, virus, and DNA vaccines, the mRNA vaccines have the advantages of encoding a plurality of proteins, not needing to enter the nucleus of cells, and rapidly producing in large quantities. However, mRNA has a large molecular weight and negative charge, leading to not entering the cells autonomously, and the mRNA is easily degraded by enzymes in the body, resulting in poor stability. The key to the development of the mRNA vaccines, therefore, lies in the development of drug-delivery systems that improve their cell entry efficiency and stability.

Lipid nanoparticle (LNP) is currently the most widely used mRNA delivery system in clinical practice. The current clinically approved LNP for delivering COVID-19 mRNA vaccine consists of four components: an ionizable lipid, a helper phospholipid, a PEGylated lipid, and cholesterol. After intramuscular injection, the mRNA vaccines can activate both humoral and cellular immune responses and produce high levels of neutralizing antibodies and killer T cells to effectively kill viruses invading the body.

However, current mRNA vaccines based on LNP carriers still face shortcomings such as strong side effects and inability to activate the mucosal immune response. Most infectious diseases, including SARS-COV-2, are transmitted through the mucosa, and activating the mucosal immune response with mRNA vaccines can cut off the transmission of pathogens from the source, which is expected to greatly shorten the transmission time of pathogens and effectively control the spread of infectious diseases. Therefore, it is expected to provide a new type of mRNA delivery carrier that can activate the mucosal immune response more effectively.

### SUMMARY

One or more embodiments of the present disclosure provide a lipid nanoparticle, the lipid nanoparticle comprises a carrier and an encapsulated nucleic acid, the carrier comprises an ionizable lipid, a helper phospholipid, a PEGylated lipid, cholesterol and derivatives thereof, and a retinoid compound; and the nucleic acid is one or more of mRNA, circRNA, siRNA, microRNA, antisense nucleic acid, and plasmid.

One or more embodiments of the present disclosure provide a preparation method for the lipid nanoparticle, comprising: obtaining the lipid nanoparticle based on an aqueous phase containing the nucleic acid and a lipid organic phase by an ethanol dilution method or a microfluidic method.

One or more embodiments of the present disclosure provide use of the lipid nanoparticle in a biological product.

One or more embodiments of the present disclosure provide a biological product comprising the lipid nanoparticle as described above.

### BRIEF DESCRIPTION OF THE DRAWINGS

The present disclosure will be further illustrated by way of exemplary embodiments, which will be described in detail by means of the accompanying drawings. These embodiments are not limiting, and in these embodiments, the same numbering denotes the same structure, wherein:

FIG. 1 are schematic diagrams of chemical structures of retinoid compounds used in Embodiment 1 according to some embodiments of the present disclosure;

FIG. 2 are schematic diagrams of components and chemical structures of a commercialized SM-102 four-component LNP according to some embodiments of the present disclosure;

FIG. 3 are schematic diagrams of hydrated particle size distribution curves and polydispersity index (PDI) of samples obtained in the Embodiment 1 according to some embodiments of the present disclosure;

FIG. 4 are schematic diagrams of a hydrated particle size distribution curve and a PDI of samples obtained in Embodiment 2 according to some embodiments of the present disclosure;

FIG. 5 are schematic diagrams of high performance liquid chromatography (HPLC) spectrums of AM80 and ATRA quantification in Embodiment 3 and corresponding peak area-concentration standard curves according to some embodiments of the present disclosure;

FIG. 6 is a schematic diagram of the average fluorescence intensity of DC2.4 cells detected by flow cytometry in Embodiment 4 according to some embodiments of the present disclosure (normalized by the SM-102);

FIG. 7 is a schematic diagram of results of bioluminescence signals detected by a microplate reader in Embodiment 5 according to some embodiments of the present disclosure (normalized by the SM-102);

FIG. 8 is a schematic diagram of results of the bioluminescence signals detected by the microplate reader in Embodiment 6 according to some embodiments of the present disclosure (normalized by each control group);

FIG. 9 is a schematic diagram of results of the bioluminescence signals detected by the microplate reader in Embodiment 6 according to some embodiments of the present disclosure (normalized by each control group);

FIG. 10 is a schematic diagram of results of the bioluminescence signals detected by the microplate reader in Embodiment 7 according to some embodiments of the present disclosure (normalized by the SM-102);

FIGS. 11A-11C show experiment results of flow cytometry in Embodiment 8 according to some embodiments of the present disclosure, FIG. 11A is a schematic diagram of a mechanism of flow cytometry for ratios of CCR9⁺T cells and α4β7⁺T cells in Embodiment 8; FIG. 11B are schematic diagrams of the flow cytometry results of the ability of three experimental groups (PBS, free ATRA, and mLuc@SM-102+ATRA) to activate the CCR9⁺T cells and the α4β7⁺T cells; FIG. 11C are schematic diagrams of the flow cytometry results of the ability of different doses of Am80 and mLuc@SM-102+Am80 to activate the CCR9⁺T cells and the α4β7⁺T cells;

FIGS. 12A-12D are schematic diagrams of the in vivo imaging results of small animals in Embodiment 9 according to some embodiments of the present disclosure, FIG. 12A is a schematic diagram of bioluminescence intensity at the injection site of three groups (PBS, mLuc@SM-102, and mLuc@SM-102+ATRA) of mice, FIG. 12B is a schematic diagram of ex vivo imaging of lymph nodes of the mice in FIG. 12A, FIG. 12C is a schematic diagram of the bioluminescence intensity at the injection site of three groups (PBS, mLuc@SM-102, and mLuc@SM-102+AM80) of mice, FIG. 12D is a schematic diagram of bioluminescence signal intensity statistics at the injection site of three groups (PBS, mLuc@SM-102, and mLuc@SM-102+AM80) of mice;

FIG. 13 is an exemplary schematic diagram of an animal experiment in Embodiment 10 according to some embodiments of the present disclosure;

FIGS. 14A and 14B are schematic diagrams of characterization results of the humoral immune response of mice at week 2 after booster immunization in Embodiment 10 according to some embodiments of the present disclosure, FIG. 14A is a schematic diagram of a titer of omicron S protein-specific IgG antibody in the serum of mice, FIG. 14B is a schematic diagram of a titer of omicron S protein-specific IgA antibody in the serum of mice;

FIGS. 15A-15C are schematic diagrams of characterization results of the cellular immune response of mice at week 2 after booster immunization in Embodiment 10 according to some embodiments of the present disclosure, FIG. 15A is a schematic diagram of a count of omicron S protein-specific CD4⁺T cells in the spleens of the mice, FIG. 15B is a schematic diagram of a count of omicron S protein-specific CD8⁺T cells in the spleens of the mice, FIG. 15C is a schematic diagram of a count of interferon type I (IFN-1)-secreting splenocytes in the spleens of the mice after antigen stimulation;

FIGS. 16A-16C are schematic diagrams of the characterization results of the mucosal immune response of mice at week 2 after booster immunization in Embodiment 10 according to some embodiments of the present disclosure, FIG. 16A is a schematic diagram of a content of tissue-resident T cells in the lung tissues of the mice, FIG. 16B is a schematic diagram of the titer of the omicron S protein-specific IgA antibody in the bronchoalveolar lavage fluid of the mice, FIG. 16C is a schematic diagram of the titer of the omicron S protein-specific IgA antibody in the feces of the mice;

FIGS. 17A-17I are schematic diagrams of results of the rotavirus infection protection experiment in suckling mice in Embodiment 11 according to some embodiments of the present disclosure, FIG. 17A is an exemplary schematic diagram of rotavirus, FIG. 17B is an exemplary schematic diagram of immunization and challenge of suckling mice, FIG. 17C is an exemplary curve of body weight change in suckling mice before challenge, FIGS. 17D and 17E are exemplary curves of intestinal IgA antibody titer in suckling mice on day 10, FIG. 17F is an exemplary curve of body weight change in suckling mice after challenge, FIG. 17G is a schematic diagram of diarrhea scores of suckling mice after challenge, FIG. 17H is a schematic diagram of the percentage of diarrhea in suckling mice after challenge, FIG. 17I is a schematic diagram of PCR results of viral load in feces of suckling mice at different times after challenge;

FIG. 18A is an exemplary schematic diagram of an animal experiment in Embodiment 12 according to some embodiments of the present disclosure, FIG. 18B is a schematic diagram of flow cytometry for characterization of intra-tumor antigen-specific killer T cells, FIG. 18C is a schematic diagram of quantitative results of flow cytometry for characterization of intra-tumor antigen-specific killer T cells, FIG. 18D is a photograph of in situ colorectal tumor after treatment, FIG. 18E is a schematic diagram of tumor weight analysis;

FIGS. 19A-19C are schematic diagrams of in situ tumor volume change and flow cytometry results of mice on day 15 after immunization in Embodiment 11 according to some embodiments of the present disclosure, FIGS. 19A and 19B are schematic diagrams of tumor volume and weight statistics, FIG. 19C is a schematic diagram of content of intratumor killer T cells;

FIGS. 20A and 20B are schematic diagrams of the in vivo imaging results of small animals in Embodiment 12 according to some embodiments of the present disclosure, FIG. 20A is a schematic diagram of in vivo bioluminescence imaging of three groups of mice (PBS, mOVA@SM-102, and mOVA@SM-102+ATRA) at different time points, FIG. 20B is a schematic diagram of the average luminescence signal intensity of the abdomens of the three groups of mice over time;

FIG. 21 is a curve of body weight change of mice after immunization with the LNP in Embodiment 12 according to some embodiments of the present disclosure;

FIG. 22 is a schematic diagram of a case section of a major organ of mice after immunization with the LNP in Embodiments 10 and 11 according to some embodiments of the present disclosure; and

FIGS. 23A and 23B are schematic diagrams of the storage stability of the LNP containing the retinoid compound according to some embodiments of the present disclosure.

### DETAILED DESCRIPTION

In order to more clearly illustrate the technical solutions of the embodiments of the present disclosure, the accompanying drawings required to be used in the description of the embodiments are briefly described below. Obviously, the accompanying drawings in the following description are only some embodiments or examples of the present disclosure, and a person of ordinary skill in the art can apply the present disclosure to other similar scenarios in accordance with these drawings without creative labor. Unless obviously obtained from the context or the context illustrates otherwise, the same numeral in the drawings refers to the same structure or operation.

As shown in the present disclosure and in the claims, unless the context clearly suggests an exception, the words “a”, “one”, “an” and/or “the” do not refer specifically to the singular and may include the plural. Generally, the terms “including” and “comprising” suggest only the inclusion of clearly identified steps and elements. The terms “including” and “comprising” do not constitute an exclusive list, and the method or apparatus may also include other steps or elements.

As used herein, “a plurality of” means two, three, or more.

As used herein, an “effective amount” may be an amount of a biological product in the present disclosure sufficient to achieve the intended use (including, but not limited to, treatment of the disease as defined above) determined according to methods available to a clinically licensed physician in the art. Determination of the effective dose for prevention and/or treatment is within the capabilities of a clinician or researcher in the field and may be altered by the intended use (in vitro or in vivo) or by the subject and disease condition being treated such as the body weight and age of the subject, general health, severity of the disease condition, mode of administration, and other factors affecting efficacy such as history of drug allergy. The specific administered dose varies depending on the selected specific biological product, the dosing regimen relied upon, whether or not it is co-administered with other medications, the timing of the administration, the tissues in which the medication is administered, and the physical delivery system hosted.

As used herein, “subject” refers to a specific human or other warm-blooded mammal. In some embodiments, the subject may include adults and infants, children, and other warm-blooded mammals including, but not limited to, non-human primates such as chimpanzees, other apes or monkeys, and other zoo animals, domesticated mammals, or laboratory animals such as cats, pigs, dogs, cows, sheep, mice, rats, and guinea pigs. In some embodiments, the subject is preferably a human.

As used herein, “Am80” and “AM80” refer to the same substance, i.e., tamibarotene.

To ameliorate the above technical problems, the present disclosure provides a novel LNP carrier that can simultaneously encapsulate nucleic acid drugs (mRNA, siRNA, microRNA, antisense nucleic acid, etc.) and a retinoid compound, and its preparation method and use thereof. Five-component LNP is formed by adding a certain proportion of the retinoid compound to the clinically used four-component LNP, and the addition of the retinoid compound not only significantly improves the cellular uptake and mRNA expression efficiency of the original carrier, but also induces the vaccine-activated immune cells to home to the mucosa and activates the mucosal immune response. The lipid carrier also improves the aqueous solubility of the retinoid compound, which solves the disadvantage of reliance on polyethylene glycol or oily solvents for the delivery of the retinoid compound. The present disclosure demonstrates that the five-component LNP is capable of activating the triple immune response of humoral, cellular, and mucosal responses after intramuscular or subcutaneous administration, which has significant application in the field of infectious disease vaccines and mucosal-related tumor vaccines.

The present disclosure provides a lipid nanoparticle (hereinafter referred to as LNP), the LNP includes a carrier and an encapsulated nucleic acid, the carrier includes an ionizable lipid, a helper phospholipid, a PEGylated lipid, cholesterol or derivatives thereof, and a retinoid compound. The nucleic acid includes, but is not limited to, one or more of mRNA, circRNA, siRNA, microRNA, antisense nucleic acid, and plasmid.

In some embodiments, the ionizable lipid accounts for 20 mol %-50 mol % of total lipids in the LNP. In some embodiments, the ionizable lipid may account for 25 mol %, 30 mol %, 35 mol %, 40 mol %, or 45 mol % of the total lipids in the LNP.

In some embodiments, the ionizable lipid is selected from one or more of the following compounds, including but not limited to: 8-[(2-hydroxyethyl) (6-oxo-6-decyloxyhexyl)amino]octanoic acid (heptadecan-9-yl) ester (SM-102), [(4-hydroxybutyl)azetidinyl]bis(hexane-6,1-diyl)bis(2-hexylhexanoate) (ALC-0315), 4(N,N-dimethylamino)butanoic acid (dioleyl) methyl ester (DLin-MC3-DMA), 3,6-bis{4-[bis(2-hydroxydodecyl)amino]butyl}piperazine-2,5-dione (cKK-E12), 9-(4-(dimethylamino)butanoyloxy)heptadecanedioic acid bis((Z)-nonyl-2-en-1-yl) ester (L319), N2,2-dioleyl-4-dimethylaminoethyl-[1,3]-dioxolane (DLin-KC2-DMA), 8-[(2-hydroxyethyl)(8-nonyloxy-8-oxooctyl)amino]octanoic acid (heptadecan-9-yl) ester (Lipid5), 1,1′- [(2-{4-[2-({2-[Bis(2-hydroxydodecyl)amino]ethyl}(2-hydroxydodecyl)amino)ethyl]piperazin-1-yl}ethyl)azabicyclo[bis(dodecane)-2-alcohol) (C12-200), (2,3 dioleoyl-propyl) trimethylammonium chloride (DOTAP), dimethyl dioctadecylammonium bromide (DDAB), and tetrakis(8-methylnonyl)3,3′,3″,3″-{[(methylazanediyl)bis(propane-3,1diyl)]bis(azatriyl)}tetrapropionate (3060i10). In some embodiments, the ionizable lipid is preferably 8-[(2-hydroxyethyl) (6-oxo-6-decyloxyhexyl)amino]octanoic acid (heptadecan-9-yl) ester (SM-102) and/or [(4-hydroxybutyl)azetidinyl]bis(hexane-6,1-diyl)bis(2-hexylhexanoate) (ALC-0315).

In some embodiments, the helper phospholipid accounts for 2 mol %-10 mol % of the total lipids in the LNP. In some embodiments, the helper phospholipid may account for 3 mol %, 4 mol %, 5 mol %, 6 mol %, 7 mol %, 8 mol %, or 9 mol % of the total lipids in the LNP.

In some embodiments, the helper phospholipid is selected from one or more of the following compounds, including but not limited to: 1,2-distearoyl-sn-glycero-3-phosphatidylcholine (DSPC), 1,2-dioleoyl-sn-glycero-3-phosphatidylcholine (DOPC), 1,2-dipalmitoyl-sn-glycero-3-phosphatidylcholine (DPPC), 2-oleoyl-1-palmitoyl acyl-sn-glycero-3-phosphatidylcholine (POPC), 1,2-dioleoyl-sn-glycero-3-phosphatidylethanolamine (DOPE), 2-oleoyl-1-palmitoyl-sn-glycero-3-phosphatidylethanolamine (POPE), 1,2-distearoyl-sn-glycero-3-phosphatidylethanolamine (DSPE), and 1,2-dipalmitoyl-sn-glycero-3-phosphatidylethanolamine (DPPE). In some embodiments, the helper phospholipid is preferably DSPC.

In some embodiments, the cholesterol and the derivatives thereof account for 10 mol %-40 mol % of the total lipids in the LNP. In some embodiments, the cholesterol and the derivatives thereof may account for 15 mol %, 20 mol %, 25 mol %, 30 mol %, or 35 mol % of the total lipids in the LNP.

In some embodiments, the cholesterol and the derivatives thereof are selected from one or more of the following compounds, including but not limited to: β-sitosterol, cholestanol, cholestanone, cholesterol, cholestenone, 7β-hydroxycholesterol, and 7α-hydroxycholesterol. In some embodiments, the cholesterol and the derivatives thereof are preferably the cholesterol.

In some embodiments, the PEGylated lipid accounts for 0.3 mol %-30 mol % of the total lipids in the LNP. In some embodiments, the PEGylated lipid may account for 0.5 mol %-2.5 mol % of the total lipids in the LNP. In some embodiments, the PEGylated lipid may account for 1.0 mol %, 1.5 mol %, or 2.0 mol % of the total lipids in the LNP.

In some embodiments, the PEGylated lipid is selected from one or more of the following compounds, including but not limited to: 1,2-dimyristoyl-rac-glycero-3-methoxypolyethylene glycol (DMG-PEG), 1,2-distearoyl-rac-glycero-3-methoxypolyethylene glycol (DSG-PEG), 1,2-dipalmitoyl-rac-glycero-3-methoxy polyethylene glycol (DPG-PEG), and 1,2-distearoyl-sn-glycero-3-phosphatidylethanolamine-methoxypolyethylene glycol (DSPE-PEG). In some embodiments, the PEGylated lipid is preferably DMG-PEG.

In some embodiments, the retinoid compound accounts for 1 mol %-35 mol % of the total lipids in the LNP. In some embodiments, the retinoid compound may account for 5 mol %, 10 mol %, 15 mol %, 20 mol %, 25 mol %, or 30 mol % of the total lipids in the LNP.

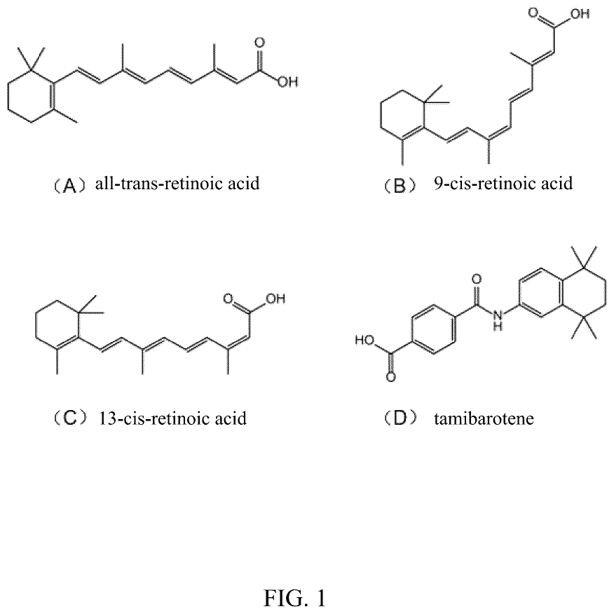

In some embodiments, the retinoid compound is one or more of retinoic acid derivatives and synthetic retinoid compounds, the retinoic acid derivatives include, but not limited to, one or more of all-trans-retinoic acid (ATRA), 9-cis-retinoic acid (9-cis-RA, 9-CRA), 13-cis-retinoic acid (13-cis-RA, 13-CRA), and the synthetic retinoid compounds include, but not limited to, retinoic acid receptor agonist such as tamibarotene (Am80). In some embodiments, the retinoid compound is preferably ATRA or Am80. The structural formulas of the compounds ATRA, 9-CRA, 13-CRA, and Am80 are shown in (A)-(D) of FIG. 1.

In some embodiments, a molar ratio of the cholesterol and the derivatives thereof to the retinoid compound is within a range of 1:2-50:1. In some embodiments, the molar ratio of the cholesterol and the derivatives thereof to the retinoid compound is within a range of 10:1-1:1. In some embodiments, the molar ratio of the cholesterol and the derivatives thereof to the retinoid compound is within a range of 4:1-1:1.

In some embodiments, the total lipids are a sum of the ionizable lipid, the helper phospholipid, the cholesterol and the derivatives thereof, the PEGylated lipid, and the retinoid compound.

In some embodiments, the nucleic acid selected of the present disclosure is selected from one or more of the following nucleic acids, including but not limited to: CpG-FAM, mRNA encoding firefly luciferase (mLuc), mRNA encoding the spike protein of the Omicron variant of SARS-COV-2 virus (S-Omicron), mRNA encoding chicken ovalbumin (mOVA), and mRNA encoding the human rotavirus strain (mHRV).

In some embodiments, an encapsulation efficiency of the nucleic acid is within a range of 30%-99%. In some embodiments, the encapsulation efficiency of the nucleic acid is within a range of 70%-90%. In some embodiments, the encapsulation efficiency of the nucleic acid is 70%, 75%, 80%, 85%, or 90%.

In some embodiments, an encapsulation efficiency of the retinoid compound is within a range of 30%-99%. In some embodiments, the encapsulation efficiency of the retinoid compound is within a range of 50%-80%. In some embodiments, the encapsulation efficiency of the retinoid compound is 50%, 60%, 65%, 70%, or 80%.

In some embodiments, a molar ratio of nitrogen element of the ionizable lipid to phosphorus element of the nucleic acid is (1-50):1. In some embodiments, the molar ratio of nitrogen element of the ionizable lipid to phosphorus element of the nucleic acid is preferably (5-20):1. According to some embodiments of the present disclosure, the molar ratio of nitrogen element of the ionizable lipid to phosphorus element of the nucleic acid is 5.67:1 or 15:1.

In some embodiments, a hydrated particle size of the LNP is within a range of 100 nm-200 nm. In some embodiments, the hydrated particle size of the LNP is within a range of 120 nm-180 nm.

In some embodiments, a molar ratio of the ionizable lipid, the helper phospholipid, the cholesterol, and the PEGylated lipid is 50:10:38.5:1.5.

In some embodiments, the carrier includes SM-102, DSPC, the cholesterol, DMG-PEG, and ATRA with a molar ratio of 36.1:7.2:27.8:1.1:27.8.

In some embodiments, the carrier includes the SM-102: the DSPC, the cholesterol, the DMG-PEG, and Am80 with a molar ratio of 36.1:7.2:27.8:1.1:27.8.

In some embodiments, the LNP includes the carrier and the encapsulated nucleic acid, the carrier includes the SM-102, the DSPC, the cholesterol, the DMG-PEG, and the ATRA with the molar ratio of 36.1:7.2:27.8:1.1:27.8, or the SM-102, the DSPC, the cholesterol, the DMG-PEG, and the AM80 with the molar ratio of 36.1:7.2:27.8:1.1:27.8; and the nucleic acid is CpG-FAM, mLuc, S-Omicron, mOVA, or mHRV.

The present disclosure also provides a preparation method of the LNP, including: obtaining the LNP based on an aqueous phase containing the nucleic acid and a lipid organic phase by an ethanol dilution method or a microfluidic method.

In some embodiments, the ethanol dilution method includes: aspirating the aqueous phase containing the nucleic acid into the lipid organic phase, mixing a liquid by rapidly aspirating the liquid with a pipette greater than 50 times, standing after mixing to obtain the LNP.

In some embodiments, the microfluidic method includes: aspirating the lipid organic phase and the aqueous phase containing the nucleic acid with a syringe, respectively, and injecting the lipid organic phase and the aqueous phase into a microfluidic chip at a flow rate of 1:3 for mixing, stopping collecting the liquid after the aqueous phase is empty in the syringe to obtain the LNP.

In some embodiments, a volume ratio of the aqueous phase containing the nucleic acid to the lipid organic phase is 3:1.

In some embodiments, the ethanol dilution method further includes dialyzing the product in a 1×PBS buffer solution with a volume of greater than 1000 times of a volume of the product for more than 4 h.

In some embodiments, the microfluidic method further includes dialyzing the collected mixture in a 1×PBS buffer solution with a volume of 1,000 times of a volume of the mixture for more than 4 h.

The present disclosure also provides use of the LNP in the preparation of a biological product.

In some embodiments, the biological product is an injectable biological product.

In some embodiments, the biological product is a vaccine. In some embodiments, the biological product is preferably an mRNA vaccine.

In some embodiments, the biological product may be selected from any of the following vaccines: an influenza vaccine, a human immunodeficiency virus (HIV) vaccine, a viral pneumonia (SARS, SARS-COV-2, etc.) vaccine, a tuberculosis vaccine, a respiratory syncytial virus (RSV) vaccine, a rotavirus (RV) vaccine, a norovirus vaccine, an enterovirus (e.g., EV71) vaccine, an intestinal mucosal-related tumor vaccine (e.g., colorectal cancer mRNA vaccine), or a lung cancer vaccine.

In some embodiments, the biological product is administered intramuscularly or subcutaneously.

In some embodiments, the biological product is used to prevent and/or treat mucosal transmission infectious diseases and mucosal-associated tumors. In some embodiments, the infectious disease may be influenza, novel coronavirus pneumonia (SARS-COV-2), atypical pneumonia (SARS), tuberculosis, bronchitis and pneumonia due to RSV infection, AIDS, diseases caused by rotaviruses, diseases caused by noroviruses, and hand-foot-and-mouth disease due to enteroviral infections. In some embodiments, the mucosal-associated tumor may be a colorectal tumor or lung cancer.

In some embodiments, the biological product is used to treat in situ colorectal cancer.

The present disclosure also provides a biological product having the meaning set forth above.

The present disclosure further provides a method of preventing and/or treating a disease, including administering a prophylactically and/or therapeutically effective amount of the biological product to the subject.

The disease is a mucosal transmission infectious disease or a mucosal-associated tumor. In some embodiments, the disease may be any one of influenza, AIDS, viral pneumonia (SARS, SARS-COV-2, etc.), tuberculosis, bronchitis and pneumonia due to RSV infection, diseases caused by rotaviruses, diseases caused by noroviruses, hand-foot-and-mouth disease due to enteroviral infections, colorectal tumors, lung cancer.

The LNP is prepared by the following method including: dissolving the ionizable lipid, the phospholipid, the PEGylated lipid, the cholesterol and the derivatives thereof, and a specific ratio of the retinoid compound in ethanol to obtain the organic phase and dissolving nucleic acid molecules in citrate buffer to obtain the aqueous phase, mixing the aqueous phase and the organic phase thoroughly, and preparing the LNP by the ethanol dilution method or the microfluidic method, and then dialyzing in the PBS buffer solution to obtain the LNP with the hydrated particle size of about 100-200 nm.

The LNP prepared in the present disclosure has a uniform particle size, good dispersion, high encapsulation efficiency, good biocompatibility, and low toxicity and side effects, which can induce the production of intestinal homing factor by T cells and activate mucosal immune response.

One or more embodiments of the present disclosure provide a preparation method for the lipid nanoparticle, including follow steps.

### (1) Preparation of the LNP

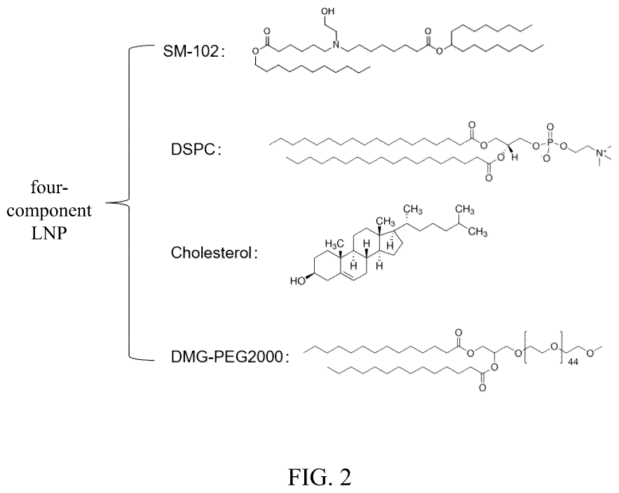

The ionizable lipid protonates to form a cationic lipid under an acidic condition, which binds to negatively charged nucleic acid by electrostatic interaction to form the nucleic acid-containing LNP. In the LNP prepared in the present disclosure, a nitrogen-to-phosphorus molar ratio of nitrogen contained in the ionizable lipid to phosphorus contained in the encapsulated nucleic acid is 5.67:1, and the molar ratio of the ionizable lipid, the helper phospholipid, the cholesterol, and the PEGylated lipid is 50:10:38.5:1.5. The ionizable lipid may be 8-[(2-hydroxyethyl) (6-oxo-6-decyloxyhexyl)amino]octanoic acid (heptadecan-9-yl) ester (SM-102); the helper phospholipid may be 1,2-distearoyl-sn-glycero-3-phosphorylcholine (DSPC); and the PEGylated lipid may be 1,2-dimyristoyl-sn-glycero-3-methoxypolyethylene glycol 2000 (DMG-PEG2000). Components and structural formulas of the commercialized SM-102 four-component lipid are shown in FIG. 2.

For the preparation of the LNP with added retinoid compound, the five-component lipid is formed by adding a certain proportion of all-trans-retinoic acid (ATRA), 9-cis-retinoic acid (9-cis-RA or 9-CRA), 13-cis-retinoic acid (13-cis-RA or 13-CRA), or AM80, etc. on the basis of the four-component LNP, and each component is dissolved in the ethanol to form the organic phase. Structural formulas of the compounds ATRA, 9-CRA, 13-CRA and Am80 are shown in (A)-(D) of FIG. 1.

CpG DNA modifying carboxy luciferin molecules (CpG-FAM) or mRNA encoding firefly luciferase protein (Firefly Luciferase mRNA, mLuc) is dissolved in 0.05 M citrate buffer to form the aqueous phase with a nucleic acid drug concentration of 0.02mg/mL-0.17 mg/mL. The LNP is prepared by the microfluidic method or the ethanol dilution method, and then dialyzed in the 1×PBS for more than 4 h.

Unless otherwise specified, the four-component LNP below includes the SM-102, the DSPC, the cholesterol, and the DMG-PEG2000 with the molar ratio of 50:10:38.5:1.5.

### (2) Preparation of the LNP With High Nitrogen-to-Phosphorus Molar Ratio

The nitrogen-to-phosphorus molar ratio of nitrogen contained in the ionizable lipid to phosphorus contained in the encapsulation nucleic acid for the LNP prepared in the present disclosure may be increased to 10:1-50:1. For the preparation of the LNP with added retinoid compound, the five-component lipid is formed by adding a certain proportion of ATRA, 9-CRA, 13-CRA or AM80, etc. on the basis of the four-component LNP, and each component is dissolved in the ethanol to form the organic phase. DNA (CpG-FAM) or mRNA (Luciferase mRNA) is dissolved in 0.05 M citrate buffer to form the aqueous phase with a nucleic acid concentration of 0.02 mg/mL-0.10 mg/mL. The LNP with high nitrogen-to-phosphorus molar ratio is prepared by the microfluidic method or the ethanol dilution method, and then dialyzed in the 1×PBS for more than 4 h.

### (3) Addition Ratio and Quantification of the Retinoid Compound in the Five-Component LNP

The addition ratio of the retinoid compound in the five-component LNP is based on the relative molar ratio to the cholesterol, and a molar ratio of the cholesterol to added retinoid compound may be within a range of 1:2-50:1. In some embodiments, the molar ratio of the cholesterol to added retinoid compound is preferably within a range of 4:1-1:1.

Unless otherwise specified below, the five-component LNP includes the SM-102, the DSPC, the cholesterol, the DMG-PEG2000, and the ATRA/AM80 with the molar ratio of 36.1:7.2:27.8:1.1:27.8.

The content of the retinoid compound in the LNP is determined by high performance liquid chromatography (HPLC), including: preparing a gradient dilution standard solution of ATRA or AM80, establishing the HPLC method, sequentially injecting 100 μL of different concentrations of the standard solution, and plotting the standard curve by using the ultraviolet absorption peak areas of different concentrations of ATRA or AM80. The five-component LNP is broken by ethanol with a volume more than 10 times the volume of the LNP, and the volume of each injection is controlled to be 100 μL; the peak area is substituted into the standard curve to obtain the content of ATRA or AM80 in the lipid solution.

### (4) Validation of the Enhanced Cellular Uptake Efficiency of the Five-Component LNP

A five-component LNP containing CpG-FAM is prepared using CpG-FAM as the encapsulated nucleic acid, and the prepared LNP is co-incubated with a dendritic cell DC2.4 cell line; an average fluorescence intensity of the cells is measured by flow cytometry to validate the cell entry efficiency of the LNP, including following steps. The five-component LNP added with the retinoid compound is used as an experimental group, and the four-component LNP is used as a control group, and the same experimental operation is performed. After DC2.4 cells are affixed to the wall and co-incubated with the LNP encapsulated with the CpG-FAM for a period of time, the average fluorescence intensity of DC2.4 cells is detected by a FITC channel of the flow cytometer to determine cellular uptake efficiency of the LNP.

### (5) Validation of the Five-Component LNP Enhancing MRNA Expression In Vitro

A mLuc-containing LNP is prepared using mLuc as the encapsulated nucleic acid; the prepared LNP is co-incubated with DC2.4 cells, and the mRNA expression efficiency of the LNP is verified by detecting the bioluminescence intensity with a microplate reader, including following steps. The five-component LNP added with the retinoid compound is used as an experimental group, and the four-component LNP is used as a control group, and the same experimental operation is performed. After DC2.4 cells are affixed to the wall and co-incubated with LNP encapsulated with the mLuc, the mRNA expression of the LNP is verified by detecting the bioluminescence intensity with the microplate reader using a firefly luciferase assay kit.

### (6) Validation of the Generalizability of Enhancing LNP MRNA Expression by the Retinoid Compound

Using the mLuc as the encapsulated nucleic acid, the nitrogen-to-phosphorus molar ratio of nitrogen contained in the ionizable lipid to phosphorus contained in the encapsulated nucleic acid is 5.67:1, the ionizable lipid is 8-[(2-hydroxyethyl)(6-oxo-6-decyloxyhexyl)amino]octanoic acid (heptadecan-9-yl) ester (SM-102), [(4-hydroxybutyl) azetidinyl]bis(hexane-6,1-diyl)bis(2-hexylhexanoate) (ALC-0315), 4(N,N-dimethylamino)butanoic acid (dioleyl)methyl ester (DLin-MC3-DMA), 3,6-bis{4-[bis(2-hydroxydodecyl)amino]butyl}piperazine-2,5-dione (cKK-E12), 9-(4-(dimethylamino)butanoyloxy)heptadecanedioic acid di((Z)-nonly-2-en-1-yl) ester (L319), respectively. Different LNPs containing the mLuc are prepared, and the prepared LNPs are co-incubated with DC2.4 cells, and bioluminescence intensity is detected using the microplate reader to verify the mRNA expression efficiency of the LNP. The above four-component LNPs containing different ionizable lipids are used as a control group, and the five-component LNPs added with the retinoid compound are used as an experimental group, and the same experimental operation is performed. After DC2.4 cells are affixed to the wall and co-incubated with the LNP encapsulated with the mLuc, the mRNA expression of the LNP is verified by detecting the bioluminescence intensity with the microplate reader using the firefly luciferase assay kit.

### (7) Validation of the Five-Component LNP for In Vitro Induction of T Cell to Express of Gut-Homing Receptors

The five-component LNP containing the mLuc is prepared using the mLuc as the encapsulated nucleic acid. T cells are sorted from mice spleen single cell suspensions using a mice T cell sorting kit. Sorted T cells are co-incubated with CD3/CD28 monoclonal antibody-coupled magnetic beads and the LNP. After 5 days of co-culture, the expression levels of C-C chemokine receptor 9 (CCR9) and α4β7 integrin on the surface of T cells are detected by flow cytometry.

### (8) Validation of the Five-Component LNP Enhancing MRNA Expression in Animals

The LNP containing mLuc is prepared using the mLuc as the encapsulated nucleic acid, and the prepared LNP is injected intramuscularly into C57BL/6 mice; the bioluminescence intensity is detected using a Small Animal In Vivo Imaging System (IVIS), including following steps. The five-component LNP added with the retinoid compound is used as an experimental group, and the four-component LNP is used as a control group, and the same experimental operation is performed. 100 μL of LNP is injected into the muscle site of the hind leg of C57BL/6 female mice, and bioluminescence imaging is performed after a period of time. Before imaging, mice are injected intraperitoneally with 200 μL of firefly luciferase substrate (D-luciferase potassium salt) at a concentration of 20 mg/mL; after 10 min of substrate injection, bioluminescence imaging is performed by the IVIS to verify the in vivo mRNA expression efficiency of the LNP.

### (9) Validation of the Five-Component LNP Simultaneously Inducing Triple Immune Responses of Humoral, Cellular, and Mucosal Immunity

The LNP containing S-Omicron is prepared using mRNA encoding the spike protein of the Omicron variant of SARS-COV-2 virus (hereinafter referred to as S-Omicron) as the encapsulated nucleic acid; the prepared LNP solution is subcutaneously injected into C57BL/6 mice for immunization to verify the prophylactic immunization effect of LNP on an infectious disease model, including following steps. The five-component LNP added with the retinoid compound is used as an experimental group, and the four-component LNP is used as a control group, and the same experimental operation is performed. The prepared LNP is injected subcutaneously into healthy C57BL/6 mice, the first injection is given at week 0, and the second injection is given at week 3 to enhance the immunity. At week 5, serum, feces, bronchoalveolar lavage fluid (BAL), lung tissue, and spleen are sampled from the mice. Humoral, cellular, and mucosal immunity related indexes are detected by enzyme-linked immunosorbent assay (ELISA), enzyme-linked immune spot analysis (ELISpot), and the flow cytometry.

### (10) Validation of the Protective Effect of the Five-Component LNP Against Mucosal Virus Infection

Using mRNA encoding human rotavirus WI61 strains VP4, VP6, and VP7 (hereinafter referred to as mHRV) as the encapsulated nucleic acid, the LNP containing mHRV is prepared, the prepared LNP solution is subcutaneously injected into BALB/C suckling mice for immunization, and then rotavirus WI61 strains is administered orally to the suckling mice for challenge to validate the protective effect of LNP against intestinal infectious diseases, including following steps. The five-component LNP added with the retinoid compound is used as an experimental group, the four-component LNP is used as a control group, and the same experimental operation is performed. BALB/c suckling mice are immunized by subcutaneous injection of LNP encapsulating mHRV in the dorsum of the neck on postnatal days 3 and 7. Three suckling mice from each group are randomly euthanized on day 10 to assess the immune response of the intestinal IgA antibody, and the rotavirus WI61 strain is administered orally to the remaining suckling mice for challenge. Mice immunized with PBS and administered orally with the WI61 strain are used as positive controls, and mice administered orally with an equal volume of PBS are used as negative controls. After the challenge, body weight changes, deaths, and diarrhea are recorded, and feces are collected to detect virus shedding.

### (11) Antitumor Effects of the Five-Component LNP in an In Situ Colorectal Tumor Model

The LNP containing mOVA is prepared using mRNA encoding chicken ovalbumin (hereinafter referred to as mOVA) as the encapsulated nucleic acid. The prepared five-component LNP is injected intramuscularly into the muscle site of the hind leg of C57BL/6 mice with a homozygous tumor in situ in the cecum (MC38-Luc-OVA), and the antitumor effects of the LNP on the in situ colorectal tumor model are verified by the IVIS and flow cytometric, including following steps. MC38-Luc-OVA is a C57BL/6 mice MC38 colon cancer cell line capable of stably expressing firefly luciferase protein and chicken ovalbumin. The five-component LNP added with the retinoid compound is used as an experimental group and the four-component LNP is used as a control group, and the same experimental operation is performed. The prepared LNP is injected into the muscle site of the hind leg of the tumor-bearing C57BL/6 mice, the first injection is given on day 3 after in situ tumor implantation, and the second injection is given on day 8 after in situ tumor implantation to enhance immunity. Tumor growth is monitored by the IVIS. Mice intestinal tumors are stripped on day 15, and the content of intra-tumor killer T cells is measured using flow cytometry.

Some embodiments of the present disclosure provide a preparation method for a novel LNP carrier simultaneously encapsulating nucleic acid drugs (mRNA, circRNA, siRNA, microRNA, the antisense nucleic acid, the plasmid) and the retinoid compound and the use thereof. The raw materials involved in the method are easily obtainable, and the method has a high encapsulation efficiency of the drugs and simple operation steps.

The five-component LNP containing the retinoid compound prepared in some embodiments of the present disclosure can significantly improve the cell entry efficiency and mRNA expression efficiency, which is universally applicable in different LNP delivery carriers. The LNP carriers simultaneously solve the difficult problem of difficult solubility of retinoic acid compounds in water and difficulty in clinical injection, realizing the synergistic function of the retinoid compound and the existing LNP carriers.

The five-component LNP containing the retinoid compound prepared in some embodiments of the present disclosure can activate humoral, cellular, and mucosal immune responses after intramuscular or subcutaneous administration, and is expected to solve the problem of the existing four-component LNP mRNA carriers failing to activate the mucosal immune response, which has great prospects for application in the field of prevention and treatment of mucosal-borne infectious diseases.

The five-component LNP containing the retinoid compound prepared in some embodiments of the present disclosure can activate T cells in subcutaneous lymph nodes and induce T cells to express gut-homing receptors after intramuscular or subcutaneous administration, increasing the infiltration of antigen-specific tumor killer cells (CD8⁺ T cells) in colorectal tumors and effectively inhibiting the growth of colorectal tumors in situ, which is expected to achieve a breakthrough in the field of colorectal tumor mRNA vaccines.

The technical solutions of the present disclosure will be described in further detail below in connection with specific embodiments. It should be understood that the following embodiments are only exemplary for illustrating and explaining the present disclosure and should not be construed as a limitation on the scope of protection of the present disclosure. Any technology realized on the basis of the foregoing contents of the present disclosure is covered by the scope of protection intended by the present disclosure.

Unless otherwise noted, the ingredients and reagents used in the following embodiments are commercially available or may be prepared by known methods.

### EMBODIMENTS

### Embodiment 1

Preparation of lipid solution: all lipids were pre-dissolved in ethanol and prepared as a stock solution for backup. The appropriate amount of DNA or mRNA was selected according to the experimental needs and the selected DNA or mRNA was diluted with 0.05 M citrate buffer to the required concentration. The dose of the used SM-102 was calculated on the basis of N/P (a nitrogen-to-phosphorus molar ratio of nitrogen contained in an ionizable lipid to phosphorus contained in an encapsulated nucleic acid) of 5.67, and only the count of nitrogen atoms of the major lipid, i.e., SM-102, was counted when calculating the nitrogen-to-phosphorus molar ratio. The amount of the remaining lipids was calculated based on a molar ratio of each lipid (SM-102: DSPC: Cholesterol: DMG-PEG-2000=50:10:38.5:1.5), and the retinoid compound (shown in FIG. 1) was added according to a molar ratio of the retinoid compound to the cholesterol of 1:2. Ethanol was added to the lipid mixture so that the final volume ratio of the nucleic acid solution to the lipid solution is 3:1.

### Preparation of LNP

(1) Preparation of LNP by an ethanol dilution method: the appropriate volume of nucleic acid stock solution was taken, and CPG-FAM or mLuc stock solution was diluted to 0.05 mg/mL (aqueous phase) with 0.05 M citrate buffer to make the final volume of 90 μL. According to a volume ratio of the nucleic acid solution to the lipid solution of 3:1, 30 μL of lipid solution (organic phase) was prepared, the two phases were mixed and then the aqueous phase was aspirated into the organic phase, a liquid was mixed by rapidly aspirating the liquid with a pipette greater than 50 times, and then left to stand for 10 min, the prepared LNP was then dialyzed in a 1×PBS buffer solution with a volume of greater than 1000 times of a volume of the LNP for more than 4 h, and then placed in a refrigerator at 4° C. for use.

(2) Preparation of LNP by a microfluidic method: the appropriate volume of the nucleic acid stock solution was taken, CPG-FAM or mLuc stock solution was diluted to 0.17 mg/mL (the aqueous phase) with 0.05 M citrate buffer to make the final volume of 300 μL. According to the volume ratio of the nucleic acid solution to the lipid solution of 3:1, a diploid volume (200 μL) of the lipid solution (organic phase) was prepared, and the two phases were mixed. The organic phase and the aqueous phase were aspirated using a 1 mL insulin needle, and the organic phase and the aqueous phase were injected into a microfluidic chip for mixing at a flow rate of 0.3 mL/min and 0.9 mL/min, respectively. The liquid was stopped collecting after the aqueous phase was empty in the insulin needle, and the prepared LNP was dialyzed in a 1×PBS buffer solution with a volume greater than 1000 times of a volume of the LNP for more than 4 h, and then placed in the refrigerator at 4° C. for use.

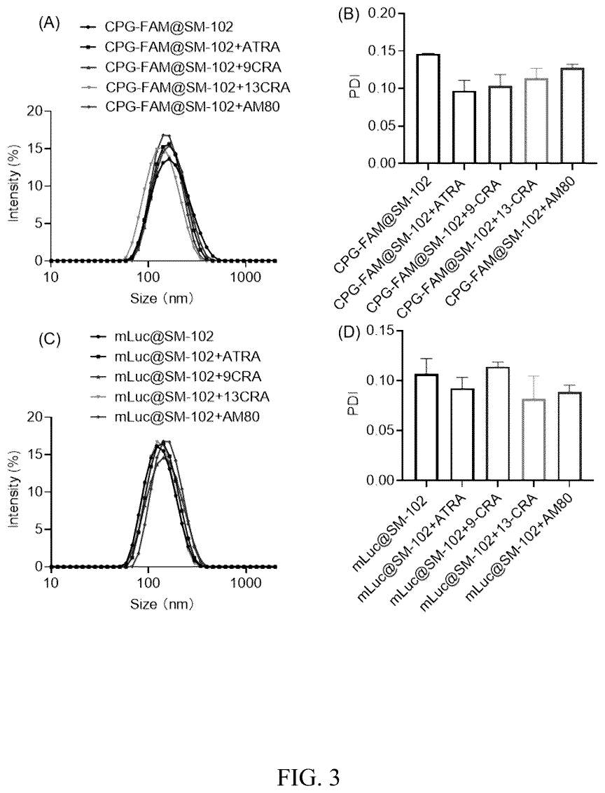

FIG. 3 shows normal distribution curves of hydrated particle sizes of prepared nanoparticles characterized by Dynamic Light Scattering (DLS), hydrated particle sizes and PDIs of CpG-FAM@SM-102, CpG-FAM@SM-102+ATRA, CpG-FAM@SM-102+9-CRA, CpG-FAM@SM-102+13-CRA, CpG-FAM@SM-102+Am80 LNP and mLuc@SM-102, mLuc@SM-102+ATRA, mLuc@SM-102+9-CRA, mLuc@SM-102+13-CRA, and mLuc@SM-102+Am80 LNP are 159.5 nm, 0.146; 151.0 nm, 0.097; 157.6 nm, 0.104; 130.1 nm, 0.114; 133.5 nm, 0.127; 123.6 nm, 0.107; 132.3 nm, 0.093; 136.6 nm, 0.114; 124.6 nm, 0.082; and 147.2 nm, 0.089.

### Embodiment 2

Lipid solution and LNP were prepared in the same manner as stated in Embodiment 1. The amount of lipid was calculated based on N/P (the nitrogen-to-phosphorus molar ratio of nitrogen contained in the ionizable lipid to phosphorus contained in the encapsulated nucleic acid) of 15/1. Only the count of nitrogen atoms was counted in the major lipid, i.e., SM-102 when calculating the nitrogen and phosphorus molar ratio. DNA or mRNA was diluted to 0.1 mg/mL with 0.05 M citrate buffer.

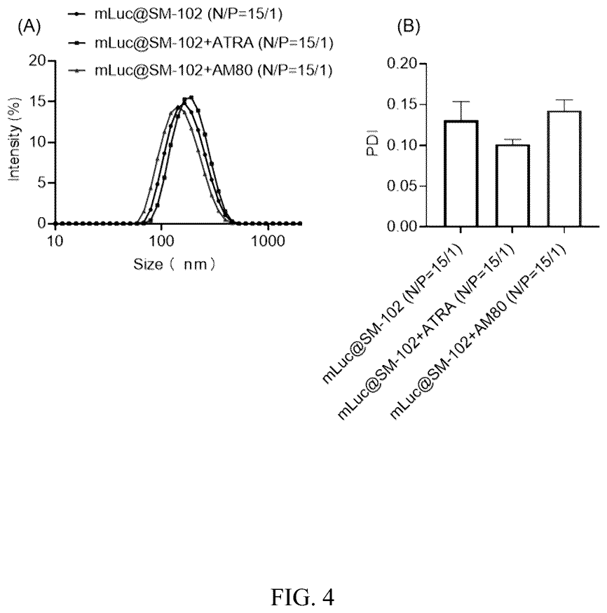

FIG. 4 shows normal distribution curves of the hydrated particle size of prepared nanoparticles characterized by DLS, hydrated particle sizes and PDIs of mLuc@SM-102 LNP, mLuc@SM-102+ATRA LNP, and mLuc@SM-102+AM80 LNP are 158.8 nm, 0.131; 173.6 nm, 0.102; and 141.7 nm, 0.143.

### Embodiment 3

Chromatographic conditions: Sunfire C18 column (150 mm×4.6 mm, 5 μm), column temperature (25° C.), mobile phase A (acetonitrile), mobile phase B (0.1 M triethylamine acetate buffer solution with a pH of 7.0), flow rate of 1.0 mL/min, and injection volume of 100 μL each time.

The standard solutions of AM80 with different concentrations were prepared with ethanol, and HPLC injection and gradient elution were performed. A proportion of the mobile phase A increased from 0% to 100% from 0 min to 15 min, with a detection wavelength of 275 nm, and the peak time of AM80 is about 10.5 min. The peak area-concentration standard curve of AM80 was obtained by integrating the absorption peak area: y=222918*x−161172 (R²=0.9992, 0<x<100 μg/mL), where x is a mass concentration of AM80 solution, and y is a corresponding AM80 absorption peak area. After diluting LNP with the ethanol, HPLC injection was performed, the AM80 content in the LNP solution was quantified according to the absorption peak area at 275 nm, and an encapsulation efficiency of AM80 is 45%-65%.

The standard solutions of different concentrations of ATRA were prepared with the ethanol, and HPLC injection and isocratic elution were performed, a ratio of mobile phase A to mobile phase B was 95:5, and the detection wavelength was 350 nm. The peak time of ATRA was about 5 min, and the peak area-concentration standard curve of ATRA was obtained by integrating the absorption peak area: y=436478*x+257220 (R²=0.9996, 0<x<200 μg/mL), where x is a mass concentration of ATRA solution, and y is a corresponding ATRA absorption peak area. After dilution of LNP with the ethanol, HPLC injection was performed, the content of ATRA in the LNP solution was quantified based on the absorption peak area at 350 nm, and an encapsulation efficiency of ATRA is 40%-70%.

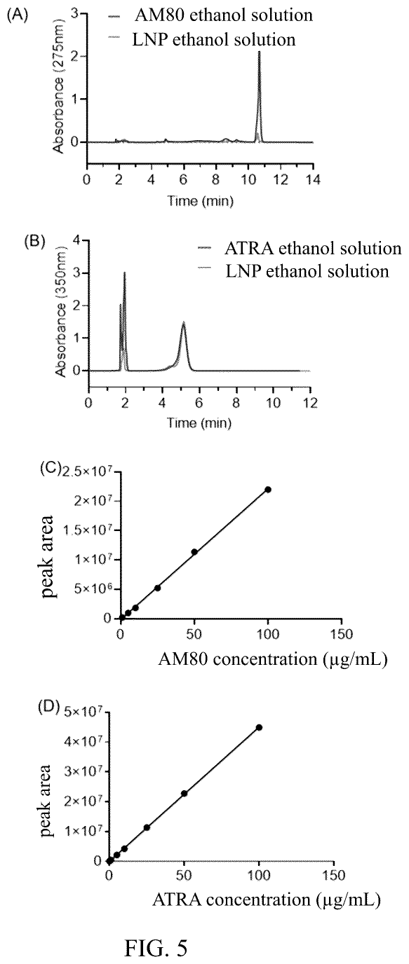

The HPLC spectra and the corresponding peak area-concentration standard curves of AM80 and ATRA are shown in (A) (B) (C) (D) of FIG. 5, respectively.

### Embodiment 4

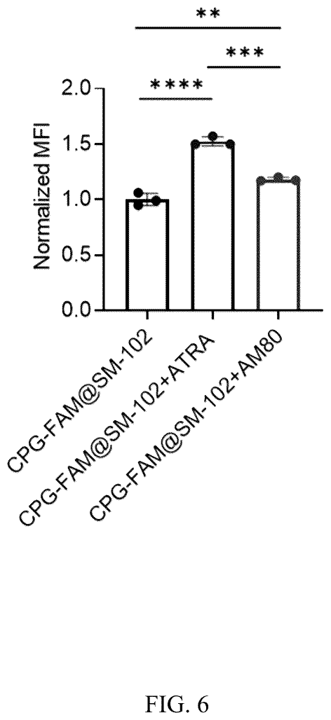

The LNP containing CpG-FAM was prepared according to the manner stated in Embodiment 1, and the five-component LNP added with the retinoid compound was used as an experimental group (CpG-FAM@SM-102+ATRA, CpG-FAM@SM-102+AM80 LNP), the four-component LNP was used as a control group (CpG-FAM@SM-102), and the same experimental operation was performed to evaluate the efficiency of the lipid nanoparticle entering cells. On the day before the experiment, DC2.4 cells were spread on the plate, and about 50,000 cells were added into each well of a 48-well cell culture plate. When the cell confluence was 70-80%, LNP was added and mixed with slight shaking, then incubated in a saturated humidity incubator with 5% CO₂ at 37° C. for 2-4 h. Cells were digested by trypsin and separated to obtain a single-cell suspension, and the average fluorescence intensity of the cells was detected using flow cytometry to validate the efficiency of the LNP uptake by DC2.4 cells. As shown in FIG. 6, the efficiency of the five-component LNP added with the retinoid compound uptake by DC2.4 cells is significantly higher than that of the four-component LNP.

### Embodiment 5

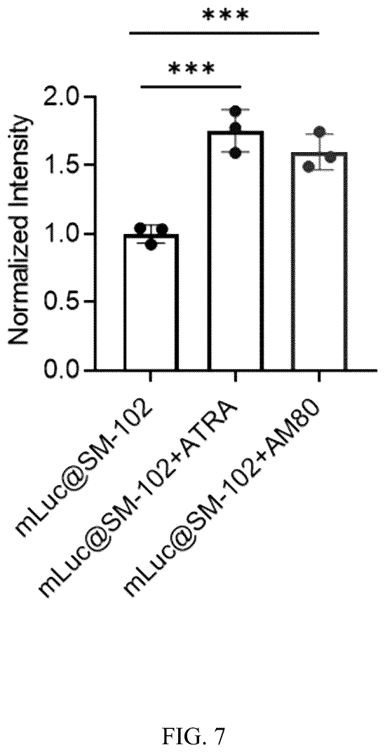

The LNP containing mLuc was prepared according to the manner stated in the Embodiment 2. The four-component LNP was used as a control group (mLuc@SM-102), and the five-component LNP with added retinoid compound was used as an experimental group (mLuc@SM-102+ATRA, mLuc@SM-102+AM80), and the prepared LNP was used for transfection of cells to test the efficiency of the LNP in delivering mRNA. On the day before transfection, DC2.4 cells were plated in 96-well cell culture plates, and about 10,000 cells were added to each well, LNP was added and mixed with slight shaking when the cell confluence was 70%-80%, and then cultured in a saturated humidity incubator with 5% CO₂ at 37° C. for 24 h. The culture plate was taken out, after the temperature of the culture plate equilibrated to room temperature, one-half of the volume of culture medium of each well was removed and 100 μL of fluorescein substrate was added to each well, and the culture plate was placed on the shaker to shake for 5 min to make the cells fully lysed, then the lysate was transferred to white 96-well plate for detecting the intensity of bioluminescence signal with the microplate reader. As shown in FIG. 7, the bioluminescence intensity of the five-component LNP added with the retinoid compound is significantly stronger than that of the four-component LNP, indicating that the five-component LNP increases the efficiency of mLuc expression in the cells.

### Embodiment 6

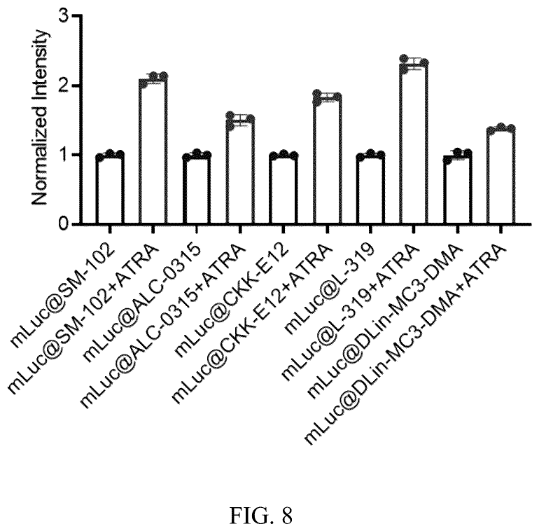

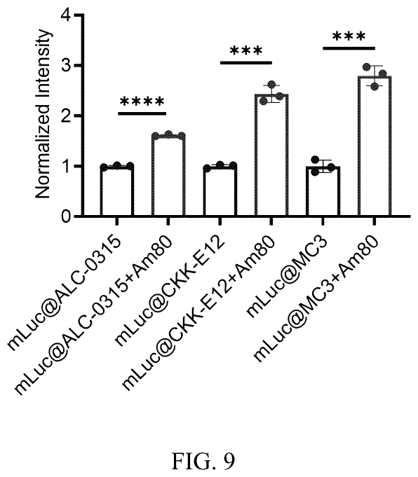

The LNP containing mLuc was prepared according to the manner of Embodiment 2, and the five-component LNP added with the retinoid compound was used as an experimental group (mLuc@SM-102+ATRA, mLuc@ALC-0315+ATRA, mLuc@CKK-E12+ATRA, mLuc@L319+ATRA, mLuc@MC3+ATRA mLuc@ALC-0315+Am80, mLuc@CKK-E12+Am80, mLuc@MC3+Am80), the four-component LNP was used as a control group (mLuc@SM-102, mLuc@ALC-0315, mLuc@CKK-E12, mLuc@L319, mLuc@MC3), and the same experimental operation was performed. The methods of detecting the efficiency of LNP in delivering mRNA, transfecting cells, and the intensity of bioluminescent signal are consistent with Embodiment 5. As shown in FIG. 8 and FIG. 9, there is a significant increase in the mRNA expression efficiency of five-component LNP added with ATRA or Am80 in each group as compared to the four-component LNP, suggesting that the five-component LNP added with the retinoid compound is generalizable in improving mRNA expression.

### Embodiment 7

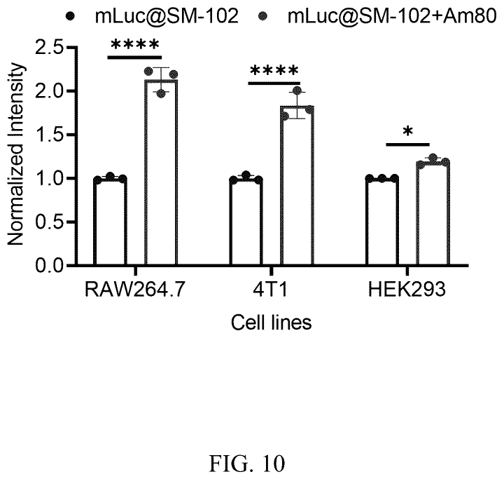

The LNP containing mLuc was prepared according to the manner of the Embodiment 2. The five-component LNP added with the retinoid compound was used as an experimental group (mLuc@SM-102+Am80), the four-component LNP was used as a control group (mLuc@SM-102), and the same experimental operation was performed. The methods of detecting the efficiency of LNP in delivering mRNA, transfecting cells, and the intensity of bioluminescent signal were consistent with Embodiment 5. The cells were replaced with mice macrophage RAW264.7, mice tumor cell 4T1, or human embryonic kidney cell HEK293. As shown in FIG. 10, the mRNA expression efficiency of each set of five-component LNP added with Am80 is significantly increased compared with that of the four-component LNP, indicating that the five-component LNP added with the retinoid compound can improve mRNA expression in different cell types.

### Embodiment 8

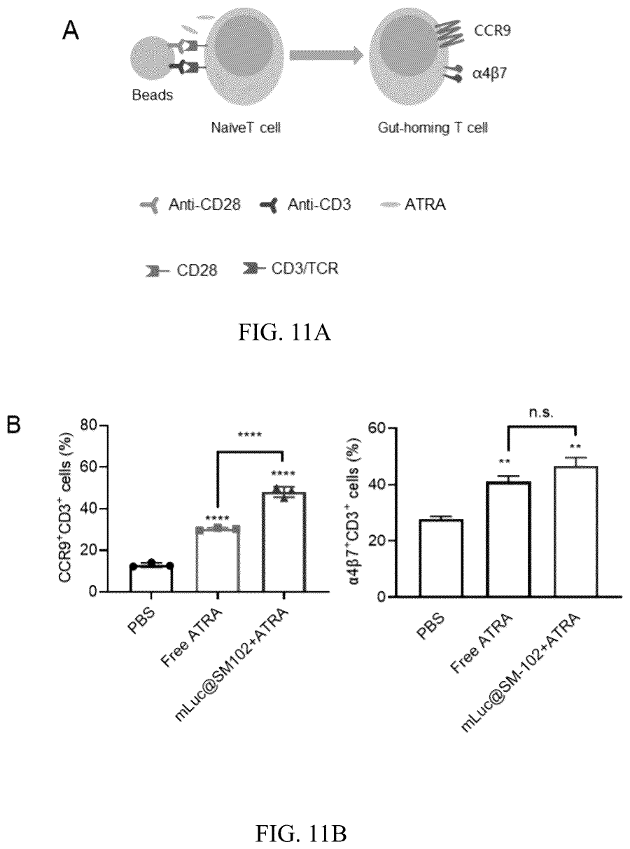

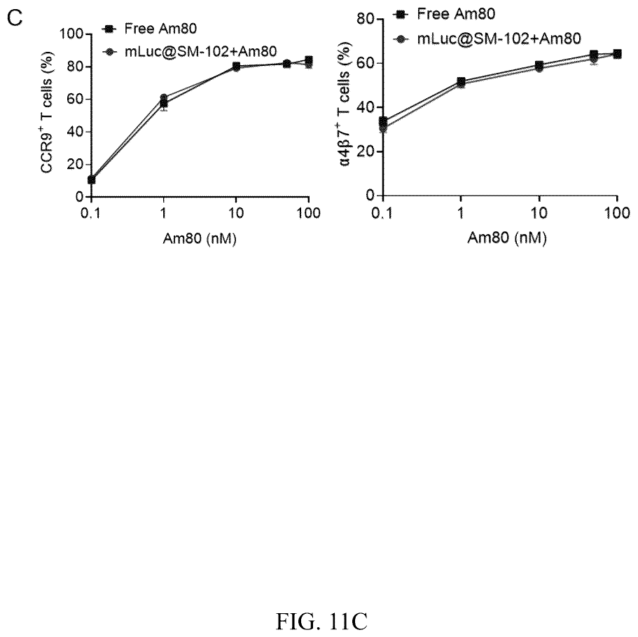

The LNP containing mLuc was prepared according to the manner of Embodiment 1. The five-component LNP (mLuc@SM-102+ATRA, mLuc@SM-102+Am80) added with the retinoid compound was used as an experimental group, the four-component LNP was used as a control group, and the same experimental operations was performed. Spleens of C57BL/6 mice were extracted and immersed in ice-cold sterile PBS buffer solution, the spleens were clamped with sterile forceps and placed on 200-mesh nylon mesh, which was placed in a petri dish containing 5 mL of ice-cold PBS buffer solution, and the mice spleen was ground with a 5 ml syringe plunger, the cell suspension was collected and centrifuged, the supernatant was discarded, and the lysed erythrocytes were washed twice with PBS buffer solution. The T cells were sorted according to the instructions of the mice T cell isolation kit. The sorted T cells were cultured using a 96-well cell culture plate with about 100,000 cells per well, and CD3/CD28 monoclonal antibody-coupled magnetic beads and LNP were added for co-culturing with the T cells. After 5 days of culture, the cells were aspirated into 1.5 mL EP tubes, washed twice with PBS buffer solution, and then stained with antibodies, and the ratios of CCR9⁺ T cells and α4β7⁺ T cells was detected using flow cytometry. As shown in FIGS. 11A-11C, the five-component LNP added with ATRA or Am80 is still effective in inducing T cells to express gut-homing receptors CCR9 and α4β7 compared to free ATRA or free Am80.

### Embodiment 9

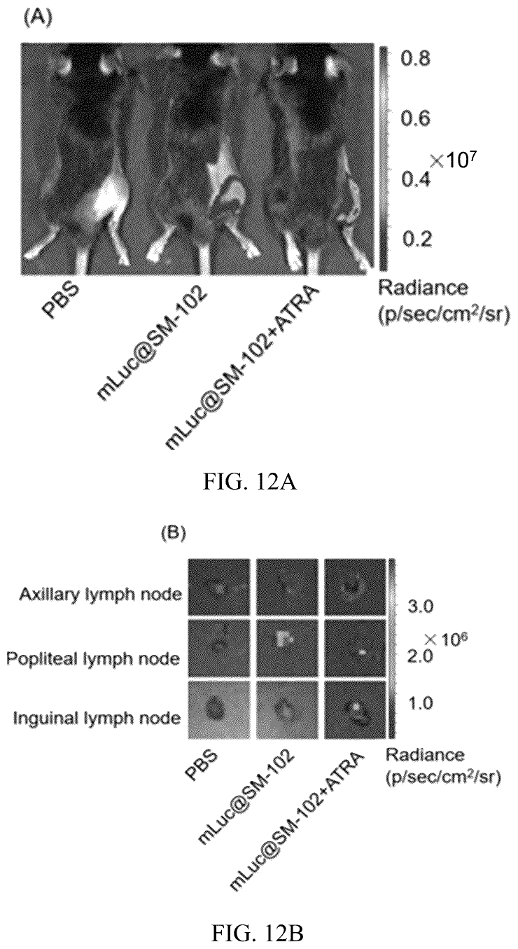

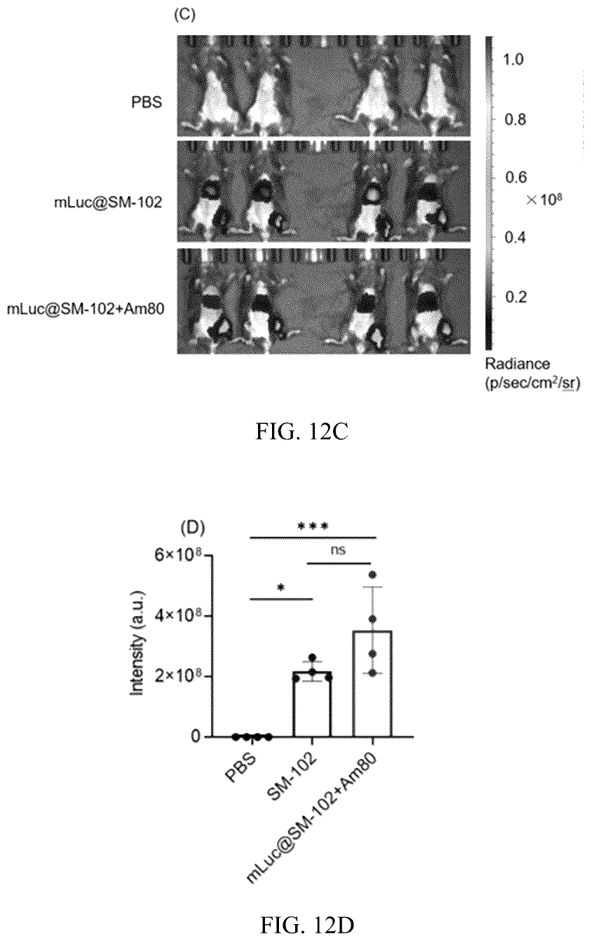

The LNP containing mLuc was prepared according to the manner of Embodiment 2. The five-component LNP added with the retinoid compound was used as an experimental group (mLuc@SM-102+ATRA, mLuc@SM-102+AM80), the four-component LNP (mLuc@SM-102) and PBS solution were used as control groups, and the same experimental operation was performed. 100 μL of LNP solution or PBS solution was injected into the muscle site of the hind leg of female C57BL/6 mice, with an administered dose of 5 μg mRNA. After 6 hours, 160 μL of 20 mg/mL luciferase substrate was injected intraperitoneally, and the in vivo bioluminescent signal was detected by IVIS after 10 min. After in vivo imaging, the mice were euthanized and the lymph nodes were extracted for in vitro bioluminescence imaging. As shown in FIGS. 12A-12D, the in vivo and ex vivo imaging results demonstrate that the efficiency of mRNA expression in vivo of the five-component LNP added with the retinoid compound is significantly higher than that of the four-component LNP.

### Embodiment 10

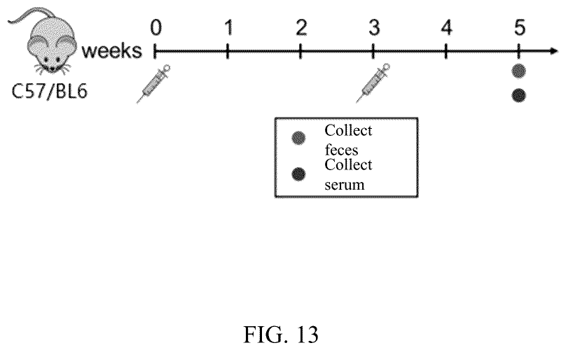

The LNP containing spike protein mRNA (S-omicron) of the omicron variant of the SARS-COV-2 virus was prepared according to the manner of Embodiment 2. The five-component LNP added with the retinoid compound was used as an experimental group (S@SM-102+AM80), a four-component LNP (S@SM-102) and a PBS solution were used as control groups, and the same experimental operations were performed. Healthy female C57BL/6 mice at 6-8 weeks were used for the experiment, with 5 mice in each experimental group. Each mouse was immunized subcutaneously at week 0 by administering a dose of LNP solution containing 20 μg of S-omicron, with 200 μg of AM80 in five-component LNP. The second booster immunization was carried out at week 3 with the same operation as the first operation, and the experimental procedure was shown in FIG. 13. Blood sampling from the orbital vein of mice was performed at week 2 after booster immunization, and serum was collected from each mouse, and the serum was assayed for the titer of the specific binding antibody against omicron spike protein, characterizing the efficiency of activation of humoral immunity.

Single cell suspensions were prepared by grinding the spleens of mice, and the splenocytes were cultured in 96-well plates with 1 million cells per well, stimulated with omicron peptide library (3 μg/mL), and protein transport inhibitors were added after 8 h of culture, and the culture was continued for 4 h. The omicron specific T cells (CD3⁺CD4⁺IFN-γ⁺, CD3⁺CD8⁺IFN-γ⁺) were measured by flow cytometry. Splenocytes were cultured using 96-well plates from the ELISPOT kit with 300,000 cells per well, stimulated with omicron spike protein (8 μg/mL), and the number of omicron-specific cells was detected after 24 h of culture according to the instructions of the kit, characterizing the efficiency of activation of cellular immunity.

The bronchoalveolar lavage (BAL) and feces of mice were taken, and the titer of omicron S protein-specific IgA antibody in BAL and feces was measured by ELISA kits, and lung monocyte suspensions were prepared by taking lung tissues from mice, and the content of tissue-resident T cells (CD69⁺CD103⁺CD8⁺) was measured in the lungs of mice by flow cytometry, which was used to characterize the efficiency of activation of mucosal immunity.

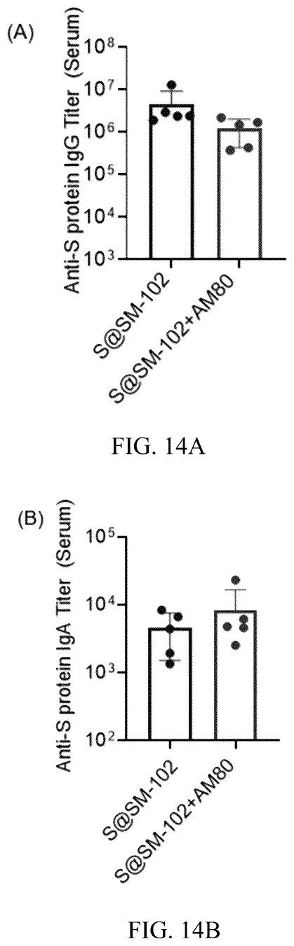

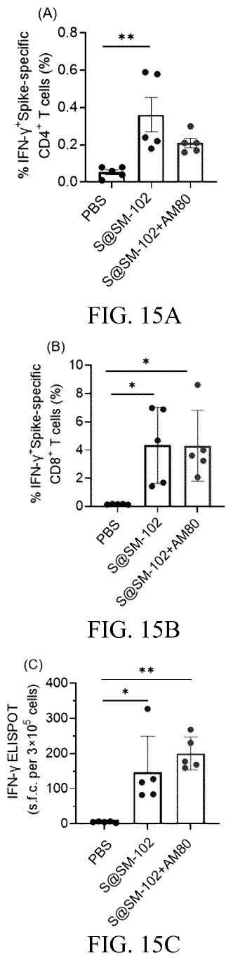

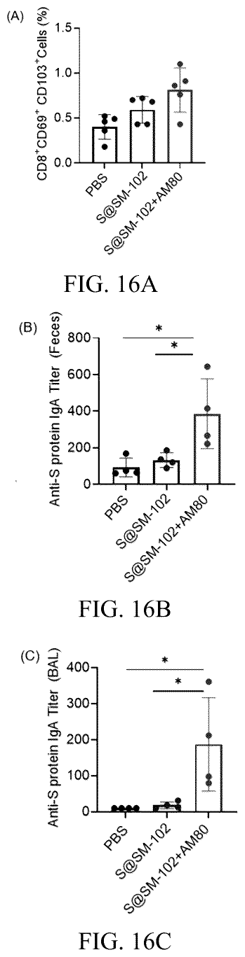

As shown in FIGS. 14A-14B, the results of serum antigen-specific IgG and IgA antibody titers indicates that compared to the PBS group, both S@SM-102 and S@SM-102+AM80 produce high levels of serum antigen-specific IgG and IgA antibody titers, indicating that both efficiently activate humoral immune response. As shown in FIGS. 15A-15C, results of flow cytometry and ELISPOT indicate that compared to the PBS group, the number of omicron-specific T cells in the spleens of mice inoculated with S@SM-102 and S@SM-102+AM80 and the number of splenocytes secreting type I interferon after antigen stimulation significantly increase, indicating that both efficiently activate the cellular immune response. As shown in FIGS. 16A-16C, the flow cytometric results of lung tissues indicate that the content of lung tissue-resident T cells increases in the S@SM-102+AM80 group compared with the PBS group and the S@SM-102 group. In BAL and feces, omicron S protein specific IgA antibody was barely detectable in the PBS and S@SM-102 groups, while the IgA antibody titers significantly increase in the S@SM-102+AM80 group, indicating that the four-component LNP fails to activate the mucosal immune response, and the five-component LNP added with the retinoid compound efficiently activates the mucosal immune response.

### Embodiment 11

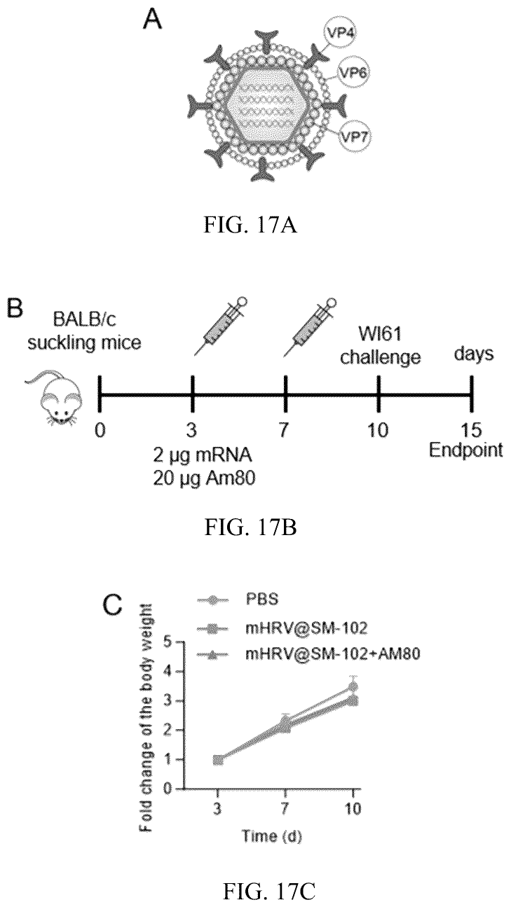

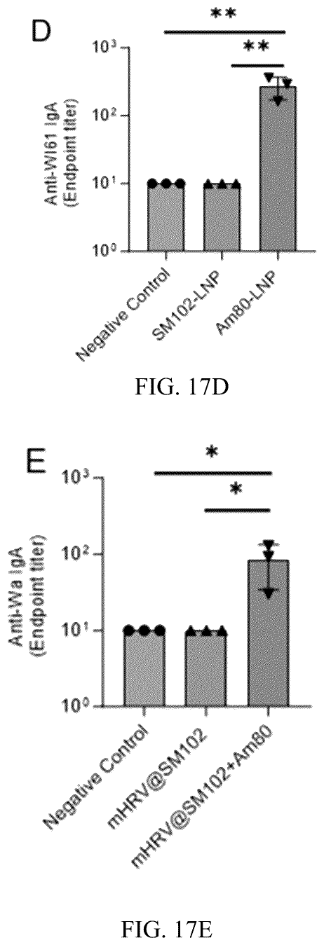

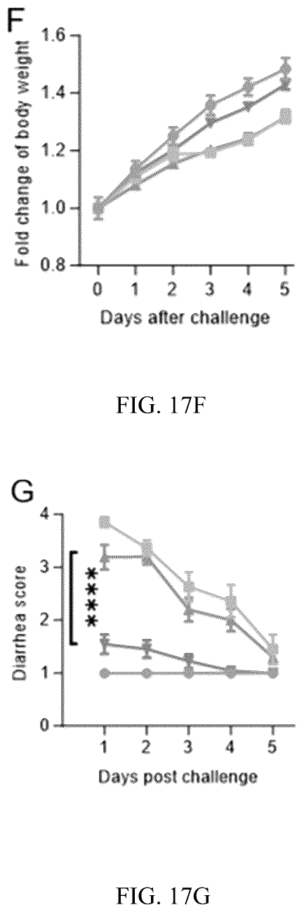

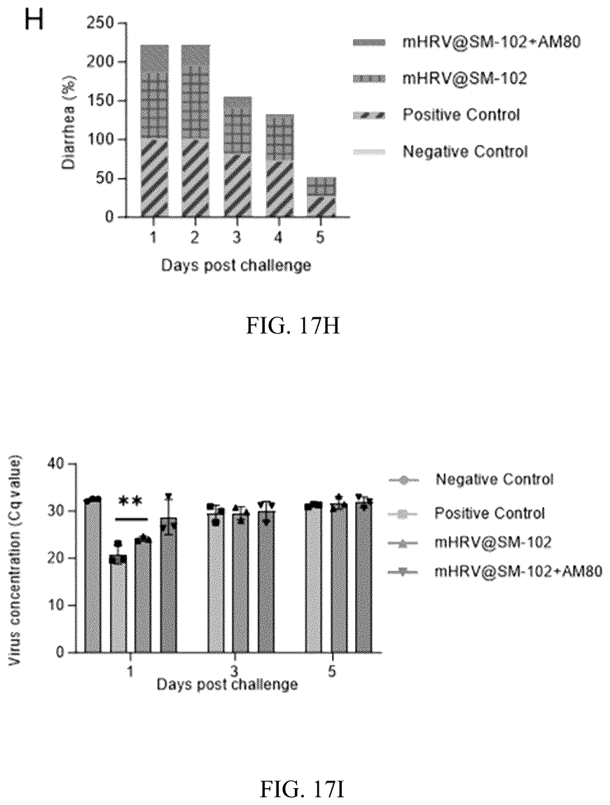

The LNPs containing mRNAs of rotavirus WI61 strains VP4, VP6, and VP7 (mHRV) were prepared according to the manner of Embodiment 2 (FIG. 17A). The five-component LNP added with the retinoid compound was used as an experimental group (mHRV@SM-102+AM80), the four-component LNP (mHRV@SM-102) and PBS solution was used as control groups, and the same experimental operation was performed. BALB/c suckling mice were immunized by subcutaneous injection on the back and the neck with LNP solution containing 2 μg of total mRNA on postnatal days 3 and 7 (FIG. 17B), and the body weights of the suckling mice were recorded to assess the potential toxicity of the vaccine. Three suckling mice in each group were randomly euthanized on day 10 to take small intestines for assessment of their intestinal IgA antibody immune response, and the remaining suckling mice were administered orally using rotavirus WI61 strain for challenge. The suckling mice immunized with PBS and administered orally with the WI61 strain were used as the positive controls, while mice administered orally with an equal volume of PBS were used as negative controls. After the challenge, weight changes, deaths, degree of diarrhea, and other indicators were recorded for each group of suckling mice, and feces were collected to detect virus shedding using Reverse Transcription-Polymerase Chain Reaction (RT-PCR) to assess the protective effect of the vaccine.

As shown in FIGS. 17A-171, vaccine immunization does not affect the body weights of the mice (FIG. 17C), and after two doses of vaccine immunization, only the intestines of the suckling mice in the Am80-LNP group produce a WI61-specific IgA antibody response, whereas there is no difference between the SM102-LNP-immunized group and the PBS group (FIG. 17D). The IgA antibody also binds to another human rotavirus Wa strain, although its titer is lower than that of the WI61 strain (FIG. 17E). Within 24 h after the challenge, 3 suckling mice die in the positive control group and 1 suckling mouse dies in the mHRV@SM-102 immunized group, and there is no death in the negative control group and the mHRV@SM-102+AM80 immunized group. Changes in body weights of suckling mice on 1-5 days after challenge are shown in FIG. 17F. Compared with the mHRV@SM-102+AM80 group, the mHRV@SM-102 immunized group shows that body weight gain of suckling mice significantly slows down after 48 h of challenge and continues until the end of the observation period. There is no statistically significant difference in body weight gain between the mHRV@SM-102+AM80 immunized group and the negative control group at any time point (P>0.05). All groups of mice experience diarrhea after the challenge, and the diarrhea of mice in each group is observed and scored. The diarrhea of mice in the mHRV@SM-102+AM80 immunized group is consistently less severe, only 36% of the mice experience diarrhea on day 1 after the challenge, their average diarrhea scores are not significantly different from those of the negative control group from day 2 after the challenge, and all mice return to normal at the end of the observation period. However, although diarrhea of mice in the mHRV@SM-102 immunized group is alleviated and the percentage of diarrhea is also decreased, there is no significant difference in the average diarrhea scores at any time point when compared to the positive control group, and more than 25% of the mice are still in diarrhea at the end of the observation period (FIGS. 17G and 17H). W161 viral RNA in the feces of suckling mice collected at different time points after the challenge was detected using real-time fluorescence quantitative PCR, as shown in FIG. 17I. The results show that the suckling mice infected with rotavirus produce a continuous detoxification process, with the maximum amount of detoxification occurring around 24 h after the challenge, while Am80-LNP immunization significantly reduces the detoxification amount at 24 h. There is no significant difference in the amount of detoxification between groups and the negative control group on day 3 and day 5 after the challenge. In conclusion, these data illustrate that the four-component LNP cannot activate mucosal immune response, and that the five-component LNP immunization with the addition of retinoid compound significantly increases the intestinal antigen-specific IgA response of the suckling mice to avoid death due to rotavirus attack, effectively reducing the degree of diarrhea and reducing the viral load in the feces.

A person skilled in the art appreciates that a variety of vaccines can be obtained by selecting encapsulated nucleic acids, for example, influenza vaccine, HIV vaccine, viral pneumonia (SARS, SARS-COV-2) vaccine, tuberculosis vaccine, respiratory syncytial virus (RSV) vaccine, norovirus vaccine, rotavirus vaccine, or the like.

### Embodiment 12

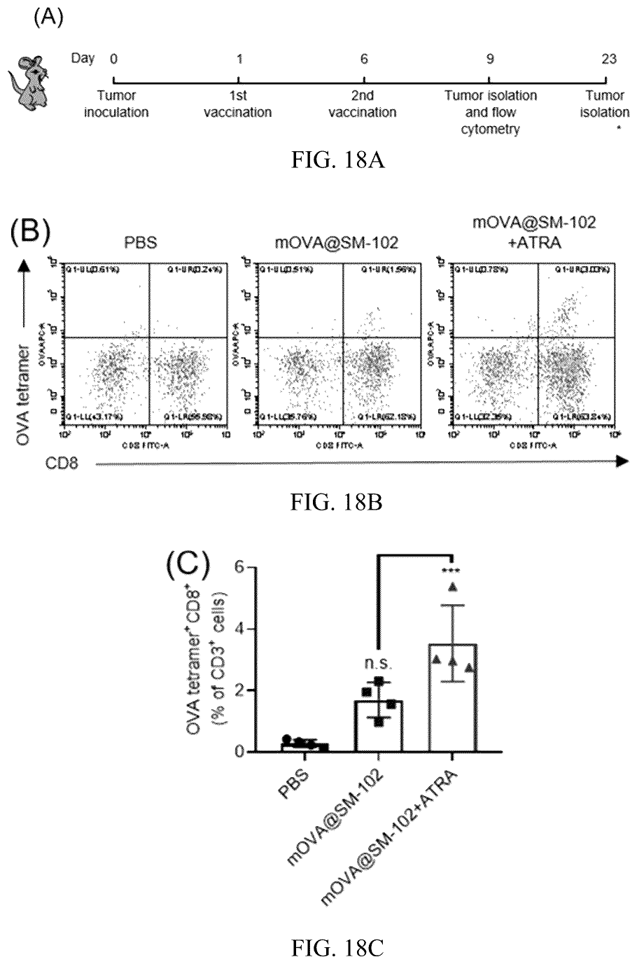

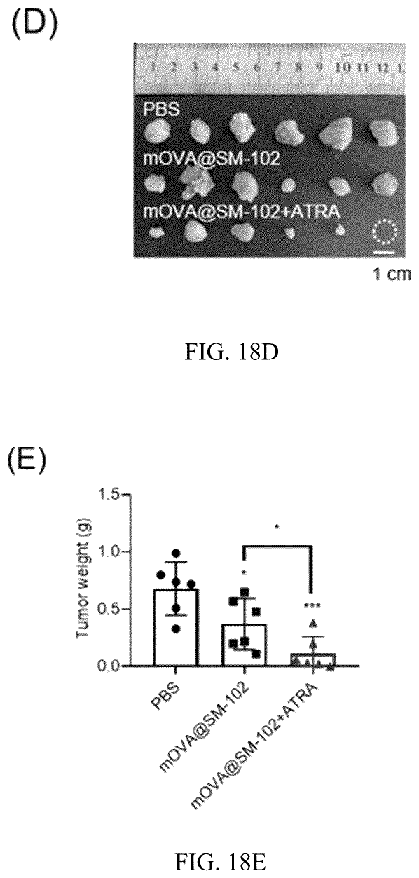

The LNP containing chicken ovalbumin mRNA (mOVA) was prepared according to the manner of Embodiment 2. The five-component LNP added with the retinoid compound (mOVA@SM-102+ATRA) was used as an experimental group, the four-component LNP (mOVA@SM-102) and PBS solution was used as the control group, and the same experimental operation was performed. Subcutaneous inoculation of the MC38-Luc-OVA cell line was performed, when the volume of the subcutaneous tumors reached about 1000 mm³, the subcutaneous tumors were peeled off and sheared into lumps of length and width of about 2 mm, and the patches were planted in the cecum of 4-6 week-old female C57BL/6 mice to construct an in situ colorectal tumor model. On day 1 after tumor implantation, the prepared LNP or PBS solution was injected into the muscle sites of the hind legs of the tumor-bearing C57BL/6 mice at a dose of 10 μg of mRNA, with 70 μg of ATRA in the five-component LNP (FIG. 18A). The second enhancement injection was performed on day 6. Mice were intraperitoneally injected with 160 μL of 20 mg/ml of D-luciferin potassium salt at different time points after tumor inoculation, respectively, and tumor growth was monitored by detecting the bioluminescence intensity of the tumors using the Small Animal IVIS. On day 9, 4 mice from each group were euthanized, the intestinal tumors were stripped, a single-cell suspension of the tumors was prepared, the number of CD3⁺CD8⁺ T cells in the tumors was detected by flow cytometry, and the tumor growth of the remaining mice continued to be monitored using the IVIS.

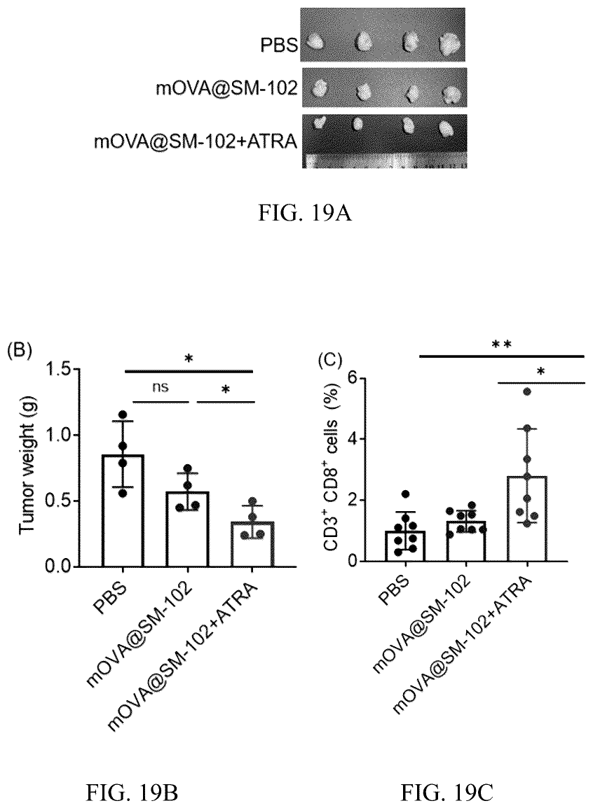

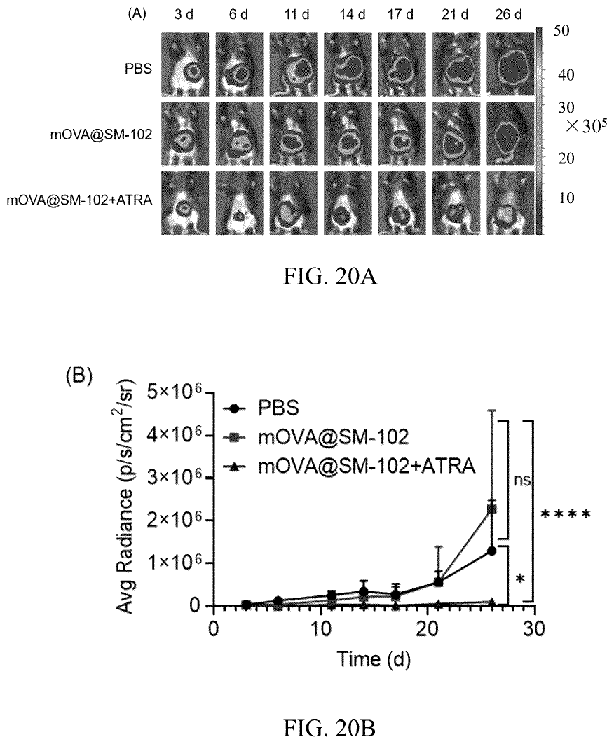

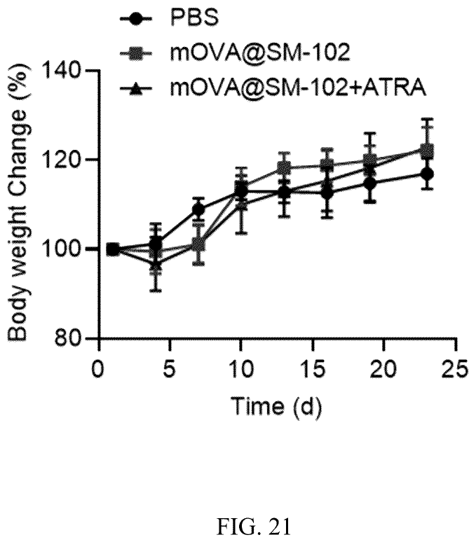

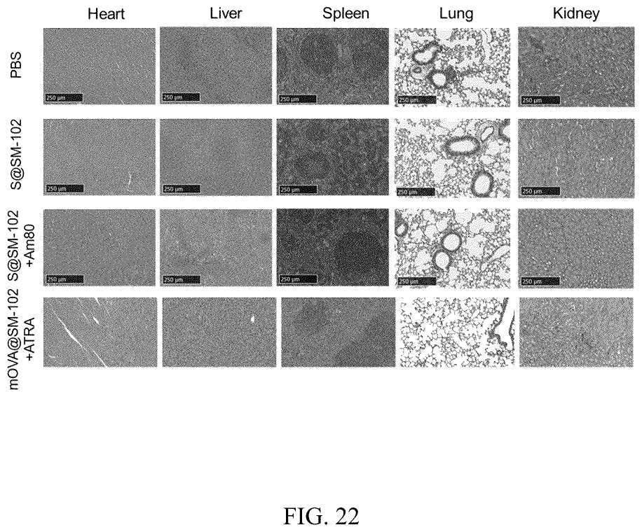

As shown in FIGS. 18A-18E and FIGS. 19A-19C, flow cytometry results indicate that compared to PBS and mOVA@SM-102, the content of intra-tumor killer T cells in mice vaccinated with the mOVA@SM-102+ATRA vaccine significantly increases, suggesting that the five-component LNP added with the retinoid compound can induce the homing of subcutaneously activated T cells in intestinal tumors, which is expected to improve the tumor suppression effect. As shown in FIGS. 18A-18E, 19A-19C, and 20A-20B, the results of tumor images and growth curves indicate that compared with PBS and mOVA@SM-102, the five-component LNP added with the retinoid compound has a significant inhibitory effect on in situ colorectal tumors in mice. As shown in FIG. 21, there is no significant change in body weight of mice after inoculation with mOVA@SM-102+ATRA and mOVA@SM-102. As shown in FIG. 22, pathologic sections of major organs show that all experimental groups do not cause damage to major organs, demonstrating good biocompatibility.

### Embodiment 13

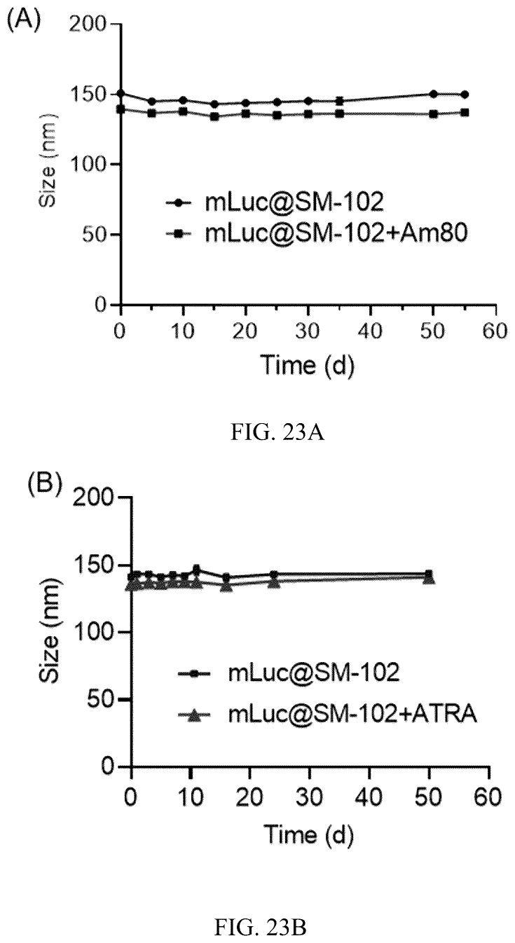

The LNP containing mLuc was prepared according to the manner of the Embodiment 2. The five-component LNP added with the retinoid compound (mLuc@SM-102+Am80, mLuc@SM-102+ATRA) was used as an experimental group, the four-component LNP (mLuc@SM-102) was used as a control group, and the same experimental operation was performed. The LNP solution was stored at 4° C. and the LNP samples were taken out at different time points to characterize the particle size using a dynamic light scatterometer. The five-component LNP is stable while stored at 4° C. as shown in FIGS. 23A and 23B.

The basic concepts have been described above, and it is apparent to those skilled in the art that the foregoing detailed disclosure is intended as an Embodiment only and does not constitute a limitation of the present disclosure. While not expressly stated herein, a person skilled in the art may make various modifications, improvements, and amendments to the present disclosure. Those types of modifications, improvements, and amendments are suggested in the present disclosure, so those types of modifications, improvements, and amendments remain within the spirit and scope of the exemplary embodiments of the present disclosure.

Also, the present disclosure uses specific words to describe embodiments of the present disclosure. For Embodiment, “an embodiment”, “one embodiment”, and/or “some embodiments” means a feature, structure, or characteristic associated with at least one embodiment of the present disclosure. Accordingly, it should be emphasized and noted that “an embodiment” or “one embodiment” or “an alternative embodiment” in different places in the present disclosure do not necessarily refer to the same embodiment. In addition, certain features, structures, or characteristics in one or more embodiments of the present disclosure may be suitably combined.

Similarly, it should be noted that in order to simplify the presentation of the disclosure of the present disclosure, and thereby aid in the understanding of one or more embodiments of the invention, the foregoing descriptions of embodiments of the present disclosure sometimes group plurality of features together in a single embodiment, accompanying drawings, or descriptions thereof. However, this method of disclosure does not imply that the objects of the present disclosure require more features than those mentioned in the claims. Rather, claimed subject matter may lie in less than all features of a single foregoing disclosed embodiment.

Some embodiments use numbers describing the number of components and attributes, and it should be understood that such numbers used in the description of embodiments are modified in some embodiments by the modifiers “approximately”, “nearly”, or “substantially”. Unless otherwise noted, the terms “approximately,” “proximately,” or “approximately” indicate that a ±20% variation in the stated number is allowed. Correspondingly, in some embodiments, the numerical parameters used in the present disclosure and claims are approximations, which can change depending on the desired characteristics of individual embodiments. In some embodiments, the numerical parameters should take into account the specified number of valid digits and employ general place-keeping methods. While the numerical domains and parameters used to confirm the breadth of their ranges in some embodiments of the present disclosure are approximations, in specific embodiments, such values are set to be as precise as practicable.

For each of the patents, patent applications, patent publication, and other materials cited in the present disclosure, such as articles, books, disclosure sheets, publications, documents, and the like, are hereby incorporated by reference in their entirety into the present disclosure. Application history documents that are inconsistent with or conflict with the contents of the present disclosure are excluded, as are documents (currently or hereafter appended to the present disclosure) that limit the broadest scope of the claims of the present disclosure. It should be noted that in the event of any inconsistency or conflict between the descriptions, definitions, and/or use of terms in the materials appended to the present disclosure and those set forth herein, the descriptions, definitions, and/or use of terms in the present disclosure shall prevail.

Finally, it should be understood that the embodiments described in the present disclosure are only used to illustrate the principles of the embodiments of the present disclosure. Other modifications may also fall within the scope of the present disclosure. As such, alternative configurations of embodiments of the present disclosure may be considered to be consistent with the teachings of the present disclosure as an embodiment, not as a limitation. Correspondingly, the embodiments of the present disclosure are not limited to the embodiments expressly presented and described herein.

What is claimed is:

1. A lipid nanoparticle, wherein the lipid nanoparticle comprises a carrier and an encapsulated nucleic acid, the carrier comprises an ionizable lipid, a helper phospholipid, a PEGylated lipid, cholesterol and derivatives thereof, and a retinoid compound; and the nucleic acid is one or more of mRNA, circRNA, siRNA, microRNA, antisense nucleic acid, and plasmid.

2. The lipid nanoparticle of claim 1, wherein the ionizable lipid accounts for 20 mol %-50 mol % of total lipids in the lipid nanoparticle;
the ionizable lipid is selected from a group including: 8-[(2-hydroxyethyl) (6-oxo-6-decyloxyhexyl)amino]octanoic acid (heptadecan-9-yl) ester (SM-102), [(4-hydroxybutyl)azetidinyl]bis(hexane-6,1-diyl)bis(2-hexylhexanoate) (ALC-0315), 4(N,N-dimethylamino)butanoic acid (dioleyl) methyl ester (DLin-MC3-DMA), 3,6-bis{4-[bis(2-hydroxydodecyl)amino]butyl}piperazine-2,5-dione (cKK-E12), 9-(4-(dimethylamino)butanoyloxy)heptadecanedioic acid bis((Z)-nonyl-2-en-1-yl) ester (L319), N2,2-dioleyl-4-dimethylaminoethyl-[1,3]-dioxolane (DLin-KC2-DMA), 8-[(2-hydroxyethyl)(8-nonyloxy-8-oxooctyl)amino]octanoic acid (heptadecan-9-yl) ester (Lipid5), 1,1′-[(2-{4-[2-({2-[bis(2-hydroxydodecyl)amino]ethyl}(2-hydroxydodecyl)amino)ethyl]piperazin-1-yl}ethyl)azabicyclo[bis(dodecane)-2-alcohol) (C12-200), (2,3 dioleoyl-propyl) trimethylammonium chloride (DOTAP), dimethyl dioctadecylammonium bromide (DDAB), and tetrakis(8-methylnonyl) 3,3′,3″,3″-{[(methylazanediyl)bis(propane-3,1diyl)]bis(azatriyl)}tetrapropionate (3060i10); and
the total lipids are a sum of the ionizable lipid, the helper phospholipid, the cholesterol and the derivatives thereof, the PEGylated lipid, and the retinoid compound.

3. The lipid nanoparticle of claim 1, wherein the helper phospholipid accounts for 2 mol %-10 mol % of the total lipids in the lipid nanoparticle; and
the helper phospholipid is selected from a group including: 1,2-distearoyl-sn-glycero-3-phosphatidylcholine (DSPC), 1,2-dioleoyl-sn-glycero-3-phosphatidylcholine (DOPC), 1,2-dipalmitoyl-sn-glycero-3-phosphatidylcholine (DPPC), 2-oleoyl-1-palmitoyl acyl-sn-glycero-3-phosphatidylcholine (POPC), 1,2-dioleoyl-sn-glycero-3-phosphatidylethanolamine (DOPE), 2-oleoyl-1-palmitoyl-sn-glycero-3-phosphatidylethanolamine (POPE), 1,2-distearoyl-sn-glycero-3-phosphatidylethanolamine (DSPE), and 1,2-dipalmitoyl-sn-glycero-3-phosphatidylethanolamine (DPPE).

4. The lipid nanoparticle of claim 1, wherein the cholesterol and the derivatives thereof account for 10 mol %-40 mol % of the total lipids in the lipid nanoparticle; and
the cholesterol and the derivatives thereof include one or more of β-sitosterol, cholestanol, cholestanone, cholesterol, cholestenone, 7β-hydroxycholesterol, and 7α-hydroxycholesterol.

5. The lipid nanoparticle of claim 1, wherein the PEGylated lipid accounts for 0.3 mol %-30 mol % of the total lipids in the lipid nanoparticle; and
the PEGylated lipid is selected from a group including: 1,2-dimyristoyl-rac-glycero-3-methoxypolyethylene glycol (DMG-PEG), 1,2-distearoyl-rac-glycero-3-methoxypolyethylene glycol (DSG-PEG), 1,2-dipalmitoyl-rac-glycero-3-methoxy polyethylene glycol (DPG-PEG), and 1,2-distearoyl-sn-glycero-3-phosphatidylethanolamine-methoxypolyethylene glycol (DSPE-PEG).

6. The lipid nanoparticle of claim 1, wherein the retinoid compound accounts for 1 mol %-35 mol % of the total lipids in the lipid nanoparticle; and
the retinoid compound is one or more of retinoic acid derivatives and synthetic retinoid compounds, the retinoic acid derivatives include one or more of all-trans-retinoic acid (ATRA), 9-cis-retinoic acid (9-cis-RA, 9-CRA), 13-cis-retinoic acid (13-cis-RA, 13-CRA), and the synthetic retinoid compounds include retinoic acid receptor agonist.

7. The lipid nanoparticle of claim 1, wherein a molar ratio of the cholesterol and the derivatives thereof to the retinoid compound is within a range of 1:2-50:1.

8. The lipid nanoparticle of claim 1, wherein an encapsulation efficiency of the nucleic acid is within a range of 30%-99%.

9. The lipid nanoparticle of claim 1, wherein an encapsulation efficiency of the retinoid compound is within a range of 30%-99%.

10. The lipid nanoparticle of claim 1, wherein a molar ratio of nitrogen element of the ionizable lipid to phosphorus element of the nucleic acid is (1-50):1.

11. The lipid nanoparticle of claim 1, wherein a hydrated particle size of the lipid nanoparticle is within a range of 100 nm-200 nm.

12. A preparation method for the lipid nanoparticle of claim 1, comprising: obtaining the lipid nanoparticle based on an aqueous phase containing the nucleic acid and a lipid organic phase by an ethanol dilution method or a microfluidic method.

13. The preparation method of claim 12, wherein the ethanol dilution method comprises: aspirating the aqueous phase containing the nucleic acid into the lipid organic phase, mixing a liquid by rapidly aspirating the liquid with a pipette greater than 50 times, standing after mixing to obtain a product, and obtaining the lipid nanoparticle.

14. The preparation method of claim 13, wherein the ethanol dilution method further comprises dialyzing the product in a 1×PBS buffer solution with a volume of greater than 1000 times of a volume of the product for more than 4 h.

15. The preparation method of claim 12, wherein the microfluidic method comprises: aspirating the lipid organic phase and the aqueous phase containing the nucleic acid with a syringe, respectively, and injecting the lipid organic phase and the aqueous phase into a microfluidic chip at a flow rate of 1:3 for mixing, stopping collecting the liquid after the aqueous phase is empty in the syringe to obtain a collected mixture, and obtaining the lipid nanoparticle.

16. The preparation method of claim 15, wherein the microfluidic method further comprises dialyzing the collected mixture in a 1×PBS buffer solution with a volume of greater than 1000 times of a volume of the mixture for more than 4 h.

17. A biological product, comprising the lipid nanoparticle of claim 1.

18. The biological product of claim 17, wherein the biological product is an injectable biological product; and
the biological product is a vaccine, preferably an mRNA vaccine.

19. The biological product of claim 17, wherein the biological product is an influenza vaccine, an AIDS (HIV) vaccine, a viral pneumonia (SARS, SARS-COV-2, etc.) vaccine, a tuberculosis vaccine, a norovirus vaccine, a rotavirus (RV) vaccine, a respiratory syncytial virus (RSV) vaccine, an enterovirus (e.g., EV71) vaccine, an intestinal mucosal-related tumor vaccine (e.g., colorectal tumor mRNA vaccine), or a lung cancer vaccine.

20. The biological product of claim 17, wherein the biological product is used for prevention and/or treatment of infectious diseases transmitted via mucosa and mucosa-related tumors; the infectious diseases are selected from influenza, novel coronavirus pneumonia, atypical pneumonia (SARS), tuberculosis, bronchitis and pneumonia caused by RSV infection, AIDS, hand-foot-and-mouth disease caused by enterovirus infection, rotavirus diseases caused by enteroviral infections, and diseases caused by noroviruses; and the mucosa-related tumors are colorectal tumors and lung cancer.
